SEQUENCE LISTING (13 sequences)

SEQ ID NO: 1 - DNA, 33 bp, Artificial Sequence
  Description of Artificial Sequence  primer
cccctgtatcctgtgtgaaattgttatccgctc

SEQ ID NO: 2 - DNA, 33 bp, Artificial Sequence
  Description of Artificial Sequence  primer
cccctgtctcctgtgtgaaattgttatccgctc

SEQ ID NO: 3 - DNA, 33 bp, Artificial Sequence
  Description of Artificial Sequence  primer
cccctgtgtcctgtgtgaaattgttatccgctc

SEQ ID NO: 4 - DNA, 33 bp, Artificial Sequence
  Description of Artificial Sequence  primer
cccctgtttcctgtgtgaaattgttatccgctc

SEQ ID NO: 5 - DNA, 24 bp, Artificial Sequence
  Description of Artificial Sequence  primer
aggacacactttaacaataggcga

SEQ ID NO: 6 - DNA, 384 bp, Artificial Sequence
  Description of Artificial Sequence  primer
cggtaatccatgggaagagatgttctatctggatattcaggccaatcttgaatcagcggaaatgcaaaaagcattgaaagagttaggggaaatcacccgttcaatgaaggtattgggctgttacccaagtgagaacgtagtgcctgttgatccaacctgatgaaaaggtgccggatgatgtgaatcatccggcactggattattactggcgattgtcattcgcctgacgcaataacacgcggctttcactctgaaaacgctgtgcgtaatcgccgaaccagaattcgagctcggtacccggggatcctctagagtcgacctgcaggcatgcaagcttggcactggccgtcgttttacaacgtcgtgactgggaaaaccctggcg

SEQ ID NO: 7 - DNA, 26 bp, Artificial Sequence
  Description of Artificial Sequence  primer
cggtaatccaattgaagagatgttct

SEQ ID NO: 8 - DNA, 23 bp, Artificial Sequence
  Description of Artificial Sequence  primer
cgccagggttttcccagtcacga

SEQ ID NO: 9 - DNA, 30 bp, Artificial Sequence
  Description of Artificial Sequence  primer
tcacttgggtaggatcccaataccttcatt

SEQ ID NO: 10 - DNA, 26 bp, Artificial Sequence
  Description of Artificial Sequence  primer
aggtattgggcgcctacccaagtgag

SEQ ID NO: 11 - DNA, 24 bp, Artificial Sequence
  Description of Artificial Sequence  primer
acccgttcaatgaaggtattgggc

SEQ ID NO: 12 - DNA, 27 bp, Artificial Sequence
  Description of Artificial Sequence  primer
aacaggcactacgttctcacttgggta

SEQ ID NO: 13 - DNA, 384 bp, Artificial Sequence
  Description of Artificial Sequence  primer
cggtaatccatgggaagagatgttctatctggatattcaggccaatcttgaatcagcggaaatgcaaaaagcattgaaagagttaggggaaatcacccgttcaatgaaggtattgggcgcctacccaagtgagaacgtagtgcctgttgatccaacctgatgaaaaggtgccggatgatgtgaatcatccggcactggattattactggcgattgtcattcgcctgacgcaataacacgcggctttcactctgaaaacgctgtgcgtaatcgccgaaccagaattcgagctcggtacccggggatcctctagagtcgacctgcaggcatgcaagcttggcactggccgtcgttttacaacgtcgtgactgggaaaaccctggcg

(19) United States
(12) Patent Application Publication
(10) Pub. No.: US 2002/0009727 A1
(43) Pub. Date: Jan. 24, 2002

(54) Detection of single nucleotide polymorphisms

(76) Inventors: Gary A. Schultz, Ithaca, NY (US); Sheng Zhang, Ithaca, NY (US); Colleen K. Van Pelt, Ithaca, NY (US)

Correspondence Address: Michael L. Goldman, NIXON PEABODY LLP, Clinton Square, P.O. Box 31051, Rochester, NY, 14603, US

(21) Appl. No.: 09/757,992

(22) Filed: Jan. 10, 2001

Related U.S. Application Data
(60) Provisional application No. 60/179,844, filed on Feb. 2, 2000.

(51) Int. Cl. C12Q 1/68; B01D 59/44

(52) U.S. Cl. 435/6; 250/285

(57) ABSTRACT

The present invention relates to a method of detecting single nucleotide polymorphisms by providing a target nucleic acid molecule, an oligonucleotide primer complementary to a portion of the target nucleic acid molecule, a nucleic acid polymerizing enzyme, and a plurality of types of nucleotide analogs. The target nucleic molecule, the oligonucleotide primer, the nucleic acid polymerizing enzyme, and the nucleotide analogs, each type being present in a first amount, are blended to form an extension solution where the oligonucleotide primer is hybridized to the target nucleic acid molecule to form a primed target nucleic acid molecule and the nucleic acid polymerizing enzyme is positioned to add nucleotide analogs to the primed target nucleic acid molecule at an active site. The oligonucleotide primer in the extension solution is extended by using the nucleic acid polymerizing enzyme to add a nucleotide analog to the oligonucleotide primer at the active site. This forms an extended oligonucleotide primer, wherein the nucleotide analog being added is complementary to the nucleotide of the target nucleic acid molecule at the active site. The amounts of each type of the nucleotide analogs in the extension solution after the extending step are then determined where each type is present in a second amount. The first and second amounts of each type of the nucleotide analog are compared. The type of nucleotide analog where the first and second amounts differ as the nucleotide added to the oligonucleotide primer at the active site is then identified. The steps of extending, determining the amounts of each type of the nucleotide analog, comparing the first and second amounts of the nucleotide analog, and said identifying the type of nucleotide analog added may be repeated, either after repeating the blending with the extended oligonucleotide primer or after determining the amounts of each type of dideoxynucleotide or dideoxynucleotide analog remaining in the extension solution as the new first amount. As a result, the nucleotide at the active site of the target nucleic acid molecule is determined. Also disclosed is an apparatus and composition for carrying out this method.

This application claims benefit of U.S. Provisional Patent Application Serial No. 60/179,844, filed on Feb. 2, 2000.

### FIELD OF THE INVENTION

The present invention relates to the detection of single nucleotide polymorphisms.

### BACKGROUND OF THE INVENTION

Single-nucleotide polymorphisms (SNPs) are the most frequent type of variation in the human genome with an estimated frequency of one to two polymorphic nucleotides per kilobase (Schafer et al., Nat Biotechnol 16: 33-9 (1998); Brookes, Gene 234: 177-86 (1999)). SNPs can serve as genetic markers for identifying disease genes by linkage studies in families, linkage disequilibrium in isolated populations, association analysis of patients and controls, and loss-of-heterozygosity studies in tumors (Wang et al., Science 280: 1077-82 (1998)). Although some SNPs in single genes are associated with heritable diseases such as cystic fibrosis, sickle cell anemia, colorectal cancer, and retinitis pigmentosa (Kerem et al., Science 245: 1073-80 (1989); Fearon et al., Cell 61: 759-67 (1990); Sung et al., Proc Natl Acad Sci USA 88: 6481-5 (1991)), most SNPs are “silent”. They can alter phenotype by either controlling the splicing together of exon from intron-containing genes or changing the way mRNA folds. Recently, there has been increasing knowledge of the genetic basis of SNPs for individual differences in drug response (McCarthy et al., Nat Biotechnol 18: 505-8 (2000); Roses, Nature 405: 857-65 (2000)). Insights into differences between alleles or mutations present in different individuals can also illuminate the interplay of environment with disease susceptibility. For example, in the p53 tumor suppressor gene, over 400 mutations have been found to be associated with tumors and used to determine individuals with increased cancer risk (Kurian et al., J Pathol 187: 267-71 (1999)). All these applications involve the analysis of a large number of samples and will eventually require rapid, inexpensive, and highly automated methods for genotyping analysis.

Because of the importance of identifying SNPs, a number of gel-based methods have been described for their detection and genotyping. These methods include single strand conformational polymorphism analysis, heteroduplex analysis, denaturing gradient gel electrophoresis, and chemical or enzyme mismatch modification assays (Schafer and Hawkins, Nat Biotechnol 16: 33-9 (1998)). To facilitate large-scale SNP identification, new technologies are being developed to replace the conventional gel-based re-sequencing methods. Perhaps the most widely employed techniques currently used for SNP identification are array hybridization assays, such as allele specific oligonucleotide microarrays in miniaturized assays (Wang, Fan et al., Science 280: 1077-82 (1998)). This approach relies on the capacity to distinguish a perfect match from a single base mismatch by hybridization of target DNA to a related set of four groups of oligonucleotides that are identical except for the base centrally located in the oligonucleotide. Mismatches in the central base of the oligonucleotide sequence have a greater destabilizing effect than mispairing at distal positions during hybridization. Thus, this strategy developed by Affymetrix utilizes a set of four oligonucleotides for each base to re-sequence. For example, a 10-kb gene requires a microarray of 40,000 oligos that can be accomplished by on-chip photolithographic synthesis (Ramsay, Nat Biotechnol 16: 40-4 (1998)). The mutation detection is based on the development of a two-color labeling scheme, in which the reference DNA is labeled with phycoerythrin (red) during the PCR amplification, while the target DNA is labeled with fluorescein (green). Both reference and target samples can then be hybridized in parallel to separate chips with identically synthesized arrays or co-hybridized to the same chip. The signal of hybridization of fluorescent products is recorded through confocal microscopy. Comparison of the images for a target sample and reference sample can yield the genotype of the target sample for thousands of SNPs being tested. By processing co-hybridization of the reference and target samples together, experimental variability during the subsequent fragmentation, hybridization, washing, and detection steps can be minimized to make array hybridization more reproducible. The interpretation of the result is based on the ratios between the hybridization signals from the reference and the target DNA with each probe (Hacia et al., Nat Genet 14: 441-7 (1996)).

Despite the impressive technology that is emerging for the hybridization to oligonucleotide arrays, potential problems with these approaches exist due to several factors. One limiting factor originates from the inherent properties of the nucleic acid hybridization. The efficiency of hybridization and thermal stability of hybrids formed between the target DNA and a short oligonucleotide probe depend strongly on the nucleotide sequence of the probe and the stringency of the reaction conditions. Furthermore, the degree of destabilization of the hybrid molecule by a mismatched base at one position is dependent on the flanking nucleotide sequence. As a result, it would be difficult to design a single set of hybridization conditions that would provide optimal signal intensities and discrimination of a large number of sequence variants simultaneously. This is particularly true for human genomic DNA which is present either in heterozygous or homozygous form. In addition, the necessity of using DNA chips composed of tens of oligonucleotide probes per analyzed nucleotide position has led to a complex setup of assays and requires mathematical algorithms for interpretation of the data.

Another popular method for high-throughput SNP analysis is called 5′ exonuclease assay in which two fluorogenic probes, double-labeled with a fluorescent reporter dye (FAM or TET) and a quencher dye (TAMRA) are included in a typical PCR amplification (Lee et al., Nucleic Acids Res 21: 3761-6 (1993); Morin et al., Biotechniques 27: 538-40, 542, 544 passim (1999)). During PCR, the allele-specific probes are cleaved by the 5′ exonuclease activity of Taq DNA polymerase, only if they are perfectly annealed to the segment being amplified. Cleavage of the probes generates an increase in the fluorescence intensity of the reporter dye. As a result, both report fluorescence that can be plotted and segregated to determine the template genotype. The advantage of this approach is to virtually eliminate post-PCR processing. However, the apparent drawbacks of this technique relate to the time and expense of establishing and optimizing conditions for each locus.

Another widely accepted method to identify SNPs is called single nucleotide primer extension (SNuPE), also known as minisequencing (Nikiforov et al., Nucleic Acids Res 22: 4167-75 (1994); Pastinen et al., Clin Chem 42: 1391-17 (1996); Landegren et al., Genome Res 8: 769-76 (1998)). This technique involves the hybridization of a primer immediately adjacent to the polymorphic locus, extension by a single dideoxynucleotide, and identification of the extended primer. An advantage of this approach, compared to hybridization with oligonucleotide probes, is that all variable nucleotides are identified with optimal discrimination using the same reaction conditions. Consequently, at least one order of magnitude of higher power for discriminating between genotyping is available using this method than with hybridization of allele-specific oligonucleotide probes in the same array format (Pastinen et al., Genome Res 7: 606-14 (1997)).

Since the first introduction of SNuPE for the identification of genetic disease (Kuppuswamy et al., Proc Natl Acad Sci USA 88: 1143-7 (1991)), several new detection methods have been developed including luminous detection (Nyren et al., Anal Biochem 208: 171-5 (1993)), calorimetric ELISA (Nikiforov et al., Nucleic Acids Res 22: 4167-75 (1994)), gel-based fluorescent assays (Pastinen et al., Clin Chem 42: 1391-7 (1996)), homogeneous fluorescent detection (Chen et al., Genet Anal 14: 157-63 (1999)), flow cytometry-based assays (Cai et al., Genomics 66: 135-43 (2000)), HPLC analysis (Hoogendoorn et al., Hum Genet 104: 89-93 (1999)). Recently, a combination of single nucleotide primer extension and matrix assisted laser desorption ionization-time of flight mass spectrometry (MALDI-TOFMS) detection has been developed (Haff et al., Genome Res 7: 378-88 (1997); Griffin et al., Trends Biotechnol 18: 77-84 (2000); Sauer et al., Nucleic Acids Res 28: E13 (2000)). This approach allows the determination of SNP sequences by measuring the mass difference between the known primer mass and the extended primer mass using MALDI-TOFMS. Discrimination of mass differences of less than 1 part in 1,000 is required to determine which of the four dideoxynucleotide triphosphate bases (ddNTPs), dideoxy-cytidine triphosphate (ddCTP), dideoxy-thymidine triphosphate (ddTTP), dideoxy-adenosine triphosphate (ddATP), and dideoxy-guanosine triphosphate (ddGTP) reacted to extend the primer. A desired capability of this technique includes the analysis of heterozygotes where two different bases are present at the same nucleotide position. The MALDI-TOFMS measurement requires the discrimination of two mass-resolved species that represent the addition of both bases complementary to those at the SNP site. This requires MALDI-TOFMS methods incorporating high mass resolution capabilities and enhanced sensitivity. Compared to the detection of a fluorescence-labeled nucleotide by non-mass spectrometric methods, mass detection is faster, and less laborious without the need for modified or labeled bases. Mass detection offers advantages in accuracy, specificity, and sensitivity. Recently, a chip-based primer extension combined with mass spectrometry detection for genotyping was performed on a 1-μL scale in the wells contained within a microchip without using conventional sample tubes and microtiter plates (Tang et al., Proc Natl Acad Sci USA 96: 10016-20 (1999)). This miniaturized method clearly provides another potential for high-throughput and low cost identification of genetic variations.

Current methods exist for the identification of SNPs using electrospray for the mass detection of the extended primers. These methods are similar to MALDI-TOFMS in that mass measurements to within 1 part in 1,000 are required to discriminate which base extended the oligonucleotide primer. Also, electrospray ionization of large oligonucleotides is difficult, requiring someone highly skilled in the interpretation of the data.

As SNPs are used in applications such as gene location, drug resistance testing, disease diagnosis, and identity testing, a concomitant increase in the rate of routine SNP characterization will be necessary. Pooling of DNA from ten to thousands of individuals into one sample before genotyping is a valuable means of streamlining large-scale SNP genotyping in disease association studies. The results from pooling are interpreted as a representation of the allele frequency distribution in the individual samples and can be used to validate a candidate SNP as common or rare or merely detect the presence of a particular variation in the pooled DNA sample. Quantitation of small molecules by electrospray ionization is well known to provide high sensitivity and linear responses over 3-4 orders of magnitude. The electrospray ionization/mass spectrometry procedure, in accordance with the present invention, can be used to accurately quantify small molecules for SNP genotyping and can provide an advantage when analyzing pooled DNA samples for the purpose of determining SNP allele frequencies.

The present invention is a single base DNA variation detection method which overcomes the above-noted deficiencies in prior techniques.

### SUMMARY OF THE INVENTION

The present invention relates to a method of detecting single nucleotide polymorphisms by providing a target nucleic acid molecule, an oligonucleotide primer complementary to a portion of the target nucleic acid molecule, a nucleic acid polymerizing enzyme, and a plurality of types of nucleotide analogs. The target nucleic acid molecule, the oligonucleotide primer, the nucleic acid polymerizing enzyme, and the nucleotide analogs, each type being present in a first amount, are blended to form an extension solution where the oligonucleotide primer is hybridized to the target nucleic acid molecule to form a primed target nucleic acid molecule and the nucleic acid polymerizing enzyme is positioned to add nucleotide analogs to the primed target nucleic acid molecule at an active site. The oligonucleotide primer in the extension solution is extended by using the nucleic acid polymerizing enzyme to add a nucleotide analog to the oligonucleotide primer at the active site. This forms an extended oligonucleotide primer where the nucleotide analog being added at the active site is complementary to the nucleotide of the target nucleic acid molecule. The amounts of each type of the nucleotide analogs in the extension solution after the extending step are then determined where each type is present in a second amount. The first and second amounts of each type of the nucleotide analog are compared. The type of nucleotide analog where the first and second amounts differ as the nucleotide added to the oligonucleotide primer at the active site is then identified. As a result, the nucleotide consumed in the primer extension reaction is determined.

Another aspect of the present invention relates to an electrospray system. This system includes an electrospray device which comprises a substrate having an injection surface and an ejection surface opposing the injection surface. The substrate is an integral monolith having an entrance orifice on the injection surface, an exit orifice on the ejection surface, a channel extending between the entrance orifice and the exit orifice, and a recess extending into the ejection surface and surrounding the exit orifice to define a nozzle on the ejection surface. The electrospray system also includes a sample preparation device positioned to transfer fluids to the electrospray device where the sample preparation device comprises a liquid passage and a metal chelating resin positioned to treat fluids passing through the liquid passage.

A further aspect of the present invention relates to an electrospray system. This system includes an electrospray device which comprises a substrate having an injection surface and an ejection surface opposing the injection surface. The substrate is an integral monolith having an entrance orifice on the injection surface, an exit orifice on the ejection surface, a channel extending between the entrance orifice and the exit orifice, and a recess extending into the ejection surface and surrounding the exit orifice to define a nozzle on the ejection surface. The electrospray system also includes a sample preparation device positioned to transfer fluids to the electrospray device where the sample preparation device comprises a liquid passage and a molecular weight filter positioned to treat fluids passing through the liquid passage.

Yet another aspect of the present invention is directed to a reagent composition which includes an aqueous carrier, an oligonucleotide primer, a mixture of nucleotide analogs of different types, magnesium acetate, a buffer, and a nucleic acid polymerizing enzyme. The oligonucleotide primer is present in the reaction mixture in molar excess while the concentration of ddNTPs is limited. In general the primer concentration is four times greater than that of each ddNTP.

Detection of the unreacted or free solution concentrations of the four ddNTPs offers many advantages over systems and methods described in the prior art. One of the main advantages is that by detecting the relative concentrations of the free ddNTPs in solution, any single-nucleotide polymorphism can be identified by only quantifying these four compounds. This greatly simplifies the detection technology required to identify SNPs.

Another advantage of the present invention is that it permits the use of double-stranded DNA. As a result, there is no need to isolate and separate single-stranded DNA. Since the process of the present invention can be carried out in solution with free primers (i.e. primers not immobilized on a solid support), improved reaction kinetics are achieved.

The present invention eliminates the complexity associated with other SNP genotyping methods described in the prior art by providing a novel primer extension reaction coupled with electrospray ionization (ESI)/mass spectrometry (MS) analysis. Nucleotide sequence variations are determined using PCR amplified double-stranded DNA without the need to use modified PCR primers and to separate and isolate single-stranded DNA. There is no requirement for complex tagging of primer extension nucleotides or nucleotide bases with, for example, radioisotope labels or fluorescent analogs. By quantifying the unreacted ddNTPs after primer extension reactions, the present invention improves the selectivity and sensitivity of prior disclosed electrospray mass spectrometry systems for the detection of SNPs. This integrates high-throughput sample preparation and analysis using primer extension reactions coupled with mass spectrometry detection. The significant demands evolving from the modem pharmacogenetics field and the growing accumulation of identified SNPs in databases requires a much faster, accurate, sensitive, and effective analytical technique to identify SNPs of individuals for drug development. As a result failed drug development efforts can be revived, patient populations can be stratified, and target genes validated. The present invention will facilitate drug development and drug discovery in the pharmaceutical industry and also be useful in other important fields such as clinical and forensic science.

Another advantage of the method of the present invention is that all extension reactions take place in solution phase without the requirement of immobilizing either the target DNA or SNP primer to a surface prior to or during primer extension. This can be achieved with great flexibility in the type of DNA being analyzed. More particularly, either single-stranded DNA or double-stranded DNA can be used without the need for a modified PCR primer to isolate a single-stranded DNA after PCR amplification.

A further advantage of the present invention is the use of electrospray mass spectrometry for the detection of these four nucleotide analogs independent of the target nucleic acid under evaluation. Mass spectrometry methods are very specific and sensitive when detecting low molecular weight molecules. The instrument and detection method may be setup to monitor four unique ion response channels, one for each nucleotide analog, to screen any target nucleic acid. The electrospray mass spectrometry method will provide for nanomolar detection sensitivity (Poon, Electrospray Ionization Mass Spectrometry pp. 499-525 (1997), which is hereby incorporated by reference), thus providing a rapid, selective and sensitive method for SNP detection.

The present invention can identify homozygous and heterozygous SNPs in the same experiment. Particularly in heterozygous cases, two bases would be near-equally reduced in concentration, while the other two bases remain unchanged in concentration. The method described in the present invention shows that each base-reduced mixture provides proportionally reduced signal intensity for the corresponding base with relatively unchanged intensity for the unreacted bases.

The extended reaction mixture, being directly analyzed by electrospray mass spectrometry, does not require complex sample preparation procedures required by other mass spectrometry-based detection methods described in the prior art, namely MALDI-TOFMS analysis (Haff et al., Genome Res 7: 378-88 (1997) and Griffin et al., Trends Biotechnol 18: 77-84 (2000), which are hereby incorporated by reference). The present invention decreases potential interference from suppression components in the extension reaction. In addition, the data analysis is less complicated due to the detection of the same four low molecular weight molecules for any SNP compared to detection of large oligonucleotides of varying composition using MALDI-TOFMS described in the prior art.

The microchip-based electrospray device of the present invention provides minimal extra-column dispersion as a result of a reduction in the extra-column volume and provides efficient, reproducible, reliable, and rugged formation of an electrospray. This electrospray device is perfectly suited as a means of electrospray of fluids from microchip-based separation devices. The design of this electrospray device is also robust such that the device can be readily mass-produced in a cost-effective, high-yielding process.

The present invention requires only one step of sample cleanup through solid phase extraction that can be miniaturized and automated by 96/384-well platform technology.

### BRIEF DESCRIPTION OF THE DRAWINGS

FIG. 1A is a schematic drawing showing the detection of simple nucleotide polymorphisms in accordance with the present invention. FIGS. 1B-D show plots of relative ion intensity versus mass spectrum response.

FIG. 2A shows a cross-sectional view of a two-nozzle electrospray device generating one electrospray plume from each nozzle for one fluid stream. FIG. 2B shows a cross-sectional view of a two-nozzle electrospray device generating 2 electrospray plumes from each nozzle for one fluid stream.

FIGS. 3A-C show devices for detecting single nucleotide polymorphisms according to the present invention. FIG. 3A shows a reaction well block for performing a reaction, such as polymerase chain reaction and primer extension. FIG. 3B shows an electrospray system which includes both the reaction well block of FIG. 3A together with an electrospray device. FIG. 3C depicts an electrospray device with individual wells to which fluid is separately provided by a movable fluid delivery probe.

FIG. 4 shows an electrospray mass spectrum of ddNTPs.

FIGS. 5A-D show the product ion mass spectra of the (M—PO₃H₂)⁻ ions of (A) ddCTP, (B) ddTTP, (C) ddATP, and (D) ddGTP.

FIGS. 6A-B are SRM MS/MS mass spectra for the (M—H)⁻ ions collisionally dissociated to the common product ion m/z 159 and for the (M—H₂PO₃)⁻ ions collisionally dissociated to the common product ion m/z 79, respectively.

FIGS. 7A-D show an electrospray mass spectrum of a solution containing 1 μM ddNTPs with the ion intensities being normalized to the same value for comparison of the ion intensity dependence on the presence or absence of magnesium from the solution on the electrospray mass spectral data. In the mass spectra, the pseudomolecular ions, (M—H)⁻, of ddCTP, ddTTP, ddATP, and ddGTP appear at m/z 450, 465, 474, and 490, respectively. In addition, the (M—PO₃H₂)⁻ ions for each of the bases, ddCTP, ddTTP, ddATP, and ddGTP, appear at m/z 370, 385, 394, and 410, respectively. FIG. 7A shows the mass spectrum of a solution containing 1 μM ddNTPs in the presence of magnesium. FIG. 7B shows the mass spectrum of a solution containing 1 μM ddNTPs with the magnesium removed using a metal chelating resin. FIG. 7C depicts the mass spectrum of a solution containing 1 μM ddNTPs with no added magnesium and eluted through a metal chelating resin. FIG. 7D shows the mass spectrum of a solution containing 1 μM ddNTPs with no added magnesium (control) and not eluted through a metal chelating resin.

FIGS. 8A-E show the SRM MS/MS mass spectra of the remaining free ddNTPs following primer extension reactions with varying SNP primer concentrations.

FIG. 9 shows the sequence of the synthetic templates (SEQ. ID. Nos. 1-4) and SNP primer (SEQ. ID. No. 5) used in detecting single nucleotide polymorphisms in accordance with the present invention. This gene is the partial lacI gene in pUC18, with 9 bases upstream (5′) from the start codon of the lacZ gene.

FIGS. 10A-E show the SRM MS/MS mass spectra of the remaining free ddNTPs following primer extension reactions which used synthetic single-stranded DNA as templates.

FIGS. 11A-E show the SRM MS/MS mass spectra of the remaining free ddNTPs following primer extension reactions. These samples represent a duplicate set to those shown in FIGS. 10A-E. The peak area ratio data for both sets of samples are provided in Table 2.

FIG. 12 shows the results from experimental work testing heterozygous cases where two polymorphic bases were present. The heterogeneous templates (equal molar mixture of two different single-stranded DNA templates) were used as targets in the primer extension reactions. All six possible combinations of heterogeneous templates were designed, and the ddNTPs expected to be consumed in the primer extension reaction for each set of templates are indicated. The templates and SNP primer were the same as in FIG. 9.

FIGS. 13A-G show the SRM MS/MS mass spectra of the remaining free ddNTPs following primer extension reactions which contained a mixture of two synthetic single-stranded DNA templates.

FIG. 14 shows the sequence of a 384 bp PCR product of partial pheA gene (SEQ. ID. No. 6) by regular PCR amplification with a mutagenic primer, W338Ipd primer (SEQ. ID. No. 7), as forward primer, #1224 primer (SEQ. ID. No. 8) as reverse primer, and pJS1 as a template. The pJS1 plasmid was constructed as described previously (Zhang et al., J Biol Chem 273: 6248-53 (1998), which is hereby incorporated by reference). The sequence of the 384 bp double-stranded PCR product as well as all amplification primers and polymorphism detection primers (SEQ. ID. Nos. 7-12) are shown. The mutagenic bases in each primer are italicized, and the bases mismatched to 384 bp DNA are underlined. For each primer, the primer binding site to one or the other strand of the target DNA sequence is indicated by a line, and the direction of DNA synthesis is indicated by an arrow. The polymorphic bases for each detection primer are shown, and the complementary bases in the target sequence for each detection primer are shown in bold.

FIGS. 15A-E show the SRM MS/MS mass spectra of the remaining free ddNTPs following extension reactions using a 384 bp double-stranded DNA PCR product as template.

FIGS. 16A-E show SRM MS/MS mass spectra of the remaining free ddNTPs following PCR extension reactions. These samples represent a duplicate set to those shown in FIGS. 15A-E.

FIG. 17 shows a 384 bp PCR product of partial pheA gene (SEQ. ID. No. 13) with a C374A mutation which was obtained by regular PCR amplification with a mutagenic primer, W338Ipd primer (SEQ. ID. No. 7), as forward primer, #1224 primer (SEQ. ID. No. 8) as reverse primer, and pSZ87 plasmid as a template (Pohnert et al., Biochemistry 38: 12212-7 (1999), which is hereby incorporated by reference). The primers are identified in FIG. 14.

FIGS. 18A-D show the SRM MS/MS mass spectra of the remaining free ddNTPs following extension reactions relating to the pheA gene with the T366pd primer (SEQ. ID. No. 11), as described with respect to FIGS. 14 and 17.

FIGS. 19A-D show the SRM MS/MS mass spectra of the remaining free ddNTPs following extension reactions relating to the pheA gene with the V383pu primer (SEQ. ID. No. 12), as described with respect to FIGS. 14 and 17.

FIGS. 20A-B show electrospray ionization/mass spectrometry (“ESI/MS”)-based primer extension genotyping dependence on single-stranded (FIG. 20A) and double-stranded (FIG. 20B) DNA template concentrations and cycle numbers. The reactions were performed at various concentrations of the synthetic single-stranded template A (SEQ. ID. No. 1) (FIG. 20A) or the 384 bp double-stranded template (SEQ. ID. No. 6) (FIG. 20B) with various thermal cycles. The other reaction reagents remained constant as described.

### DETAILED DESCRIPTION OF THE INVENTION

The present invention relates to a method of detecting single nucleotide polymorphisms by providing a target nucleic acid molecule, an oligonucleotide primer complementary to a portion of the target nucleic acid molecule, a nucleic acid polymerizing enzyme, and a plurality of types of nucleotide analogs. The target nucleic acid molecule, the oligonucleotide primer, the nucleic acid polymerizing enzyme, and the nucleotide analogs, each type being present in a first amount, are blended to form an extension solution where the oligonucleotide primer is hybridized to the target nucleic acid molecule to form a primed target nucleic acid molecule and the nucleic acid polymerizing enzyme is positioned to add nucleotide analogs to the primed target nucleic acid molecule at an active site. The oligonucleotide primer in the extension solution is extended by using the nucleic acid polymerizing enzyme to add a nucleotide analog to the oligonucleotide primer at the active site. This forms an extended oligonucleotide primer where the nucleotide analog being added is complementary to the nucleotide of the target nucleic acid molecule at the active site. The amounts of each type of the nucleotide analogs in the extension solution after the extending step are then determined where each type is present in a second amount. The first and second amounts of each type of the nucleotide analog are compared. The type of nucleotide analog where the first and second amounts differ as the nucleotide added to the oligonucleotide primer is then identified. As a result, the nucleotide at the active site of the target nucleic acid molecule is determined.

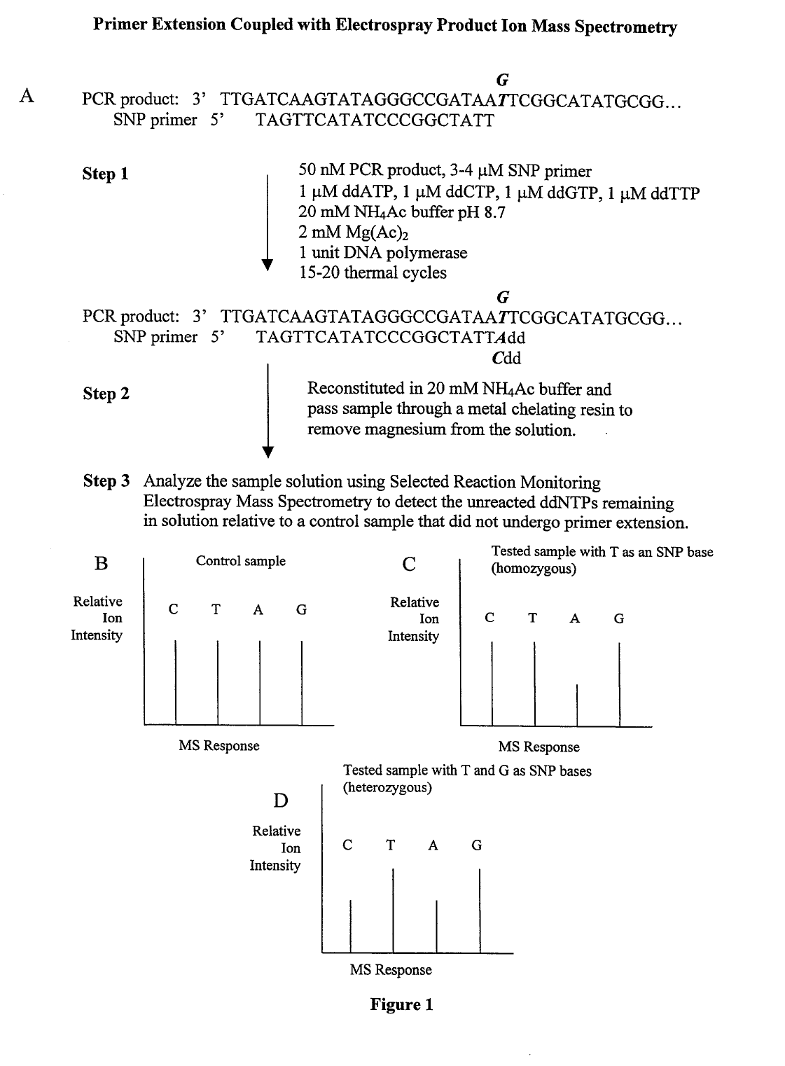

FIG. 1A is a schematic drawing showing the detection of single nucleotide polymorphisms in accordance with the present invention. After a sample is subjected to PCR amplification to increase the quantity of target nucleic acid molecule available to be detected, the PCR product is blended in Step 1 with a SNP primer complementary to a portion of the target nucleic acid sequence, an equimolar mixture of four nucleotide analogs (i.e. dideoxynucleotide triphosphates (ddNTPs), ddCTP, ddTTP, ddATP, and ddGTP), a DNA polymerase, and other reagents to form the extension solution. For example, as shown in FIG. 1, the extension solution may contain 5-50 nM of PCR product, 3-4 μM of SNP primer, 1 μM each of the ddATP, ddCTP, ddGTP, and ddTTP nucleotide analogs, 20 mM NH₄Ac buffer at a pH of 8.7, 2 mM Mg(Ac)₂, and 1 unit of DNA polymerase. A single nucleotide analog is added to the primers that are specifically designed to anneal to the target region of the PCR amplified genomic DNA fragment. Once formed, the extension solution is subjected to 15 to 20 cycles to permit the base added to the 3′ end of the SNP primer to be that which is complementary to the corresponding base in the target nucleotide. The amplified DNA template covers the known SNP variations that are located immediately at the 3′ end of the annealing primers.

The dideoxynucleotide base(s) complementary to the SNP base(s) is substantially consumed (removed) from the solution during this reaction. For homozygous SNPs, only one base is substantially consumed whereas for heterozygous SNPs, two bases are essentially consumed equally during the thermal cycle extension reaction. In FIG. 1A, the base in the target nucleic acid sequence which is susceptible to a single nucleotide polymorphism is either a T or a G. After the primer is extended by one base, as noted above, the extension solution is passed through a metal chelating resin to remove any magnesium from the solution in Step 2. The complementary base which is added to the primer is then determined by passing the extension solution as well as a control sample through an electrospray device and subjecting the electrospray to mass spectroscopy, as set forth in Step 3.

This procedure can be used to quantify the concentrations of unreacted ddNTPs remaining in each sample. The advantage of this method is the simplified analysis of the same four analytes used for all possible SNPs. Quantification of free ddNTPs after SNP primer extension reactions may be made by several approaches including but not limited to fluorescence, ion conductivity, liquid chromatography, capillary electrophoresis, mass spectrometry, nuclear magnetic resonance, colorimetric ELISA, immuno-radioactivity (IRA), radioactivity, or any combination thereof. Measurement of the unreacted nucleotide analog concentrations remaining in the reagent solution after primer extension relative to those in a control experiment allows for the immediate determination of the complementary base of the target DNA immediately adjacent to the 3′ end of the oligonucleotide primer.

Preferably, as shown in Step 3, using mass spectroscopy, the relative ion intensity for each of the nucleotide analogs is determined for each sample. By comparing the relative ion intensity of the extension solution and the control sample, the complementary base can be determined. In particular, that base is the base present in the extension solution in an amount which is less than that present in the control sample. As shown in FIG. 1B, the control sample has equal relative intensities for each of the nucleotide analogs. When the sample is homozygous for the target nucleic acid sequence with a T at the polymorphism site, the relative intensity for the complementary base, A, is lower than for the other nucleotide analogs, as shown in FIG. 1C. On the other hand, when the sample is heterozygous for the target nucleic acid sequence with a T and G at the polymorphism site, the relative intensity for the complementary bases, A and C, respectively, is lower than for the other nucleotide analogs, as shown in FIG. 1D.

In carrying out the method of the present invention, genomic DNA can be extracted from whole blood, buccal epithelial cells, and saliva stain samples which are extracted by an alkaline method (Sweet et al., Forensic Sci Int 83: 167-77 (1996); Lin et al., Biotechniques 24: 937-40 (1998); Rudbeck et al., Biotechniques 25: 588-90, 592 (1998), which are hereby incorporated by reference). For blood, 5 μL of blood with 20 μL 0.2 M NaOH are incubated at room temperature for 5 min. For an air-dried mouth swab, a proportion of the cotton is transferred to a tube, 20 μL of 0.2 M NaOH are added, and incubation is carried out at 75° C. for 10 min. This extraction procedure is carried out by adding 180 μL 0.04 M Tris-HCl pH7.5. 5 μL of the above solution is sufficient for a subsequent 50 μL PCR reaction.

PCR products are made from the target DNA by subjecting 50 μL PCR samples to treatment using an Expand PCR kit from Boehringer. The reaction mixture can contain 0.2 mM dNTPs, 0.5 μM forward and reverse primers, and 20-100 ng of genomic DNA as the template. The PCR procedure may be conducted at 95° C. for 1 min, 55° C. for 1 min, and 72° C. for 30 sec for 30-35 PCR cycles. The resulting PCR products are directly purified using a QIAGEN micro-column or Millipore Microcon-50 filter unit and further used for the later primer extension step.

The reaction mixtures for primer extension can contain 3-4 μM SNP primer, 1 μM dideoxynucleotides (ddNTPs), and 50 nM synthetic single-stranded DNA or double-stranded PCR product as the target sequence. A reaction buffer (e.g., 25 mM ammonium acetate pH 9.3) with 2 mM magnesium acetate and 1 unit of Thermosequenase may be used for the primer extension reaction. The reaction mixture (10-50 μL) can be thermally cycled at 95° C. for 30 sec, 50° C. for 60 sec, and 72° C. for 10 sec for 20 cycles in a GeneAmp PCR System 9700 instrument. This solution-based assay is readily amenable to miniaturization.

The extension reaction samples are preferably passed through a micro metal chelating gel column (e.g., immobilized iminodiacetic acid gel from PIERCE) to remove magnesium from the reaction mixture. The resulting samples then can be either directly used for MS analysis or evaporated and reconstituted into distilled water for electrospray mass spectrometry detection of the four ddNTPs.

The electrospray/mass spectrometery procedure is carried out so that the samples are analyzed in the negative ion mode. Selected reaction monitoring (“SRM”) mass spectrometry/mass spectrometry (“MS/MS”) experiments monitor unique precursor-product ion transitions for each ddNTP. For ddCTP, the SRM transition is either m/z 450→m/z 159 or m/z 370→m/z 79. For ddTTP, the SRM transition is either m/z 465→m/z 159 or m/z 385→m/z 79. For ddATP, the SRM transition is either m/z 474→m/z 159 or m/z 394→m/z 79. For ddGTP, the SRM transition is either m/z 490→m/z 159 or m/z 410→m/z 79. The relative concentration of the ddNTPs in each sample is compared to a non-extended reaction control. The base(s) complementary to the consumed ddNTPs during the primer extension reaction can be assigned as the SNP base for both homozygous and heterozygous alleles based upon the relative ion responses of each of the four ddNTPs.

Nucleotide analogs which are useful in carrying out the present invention by serving as substrate molecules for the nucleic acid polymerizing enzyme include dNTPs, NTPs, modified dNTPs or NTPs, peptide nucleotides, modified peptide nucleotides, or modified phosphate-sugar backbone nucleotides.

The process of the present invention can be used to determine the single nucleotide variations of any nucleic acid molecule, including RNA, double-stranded or single-stranded DNA, single stranded DNA hairpins, DNA/RNA hybrids, RNA with a recognition site for binding of the polymerase, or RNA hairpins.

The oligonucleotide primer used in carrying out the process of the present invention can be a ribonucleotide, deoxyribonucleotide, modified ribonucleotide, modified deoxyribonucleotide, peptide nucleic acid, modified peptide nucleic acid, modified phosphate-sugar backbone oligonucleotide, and other nucleotide and oligonucleotide analogs. It can be either synthetic or produced naturally by primases, RNA polymerases, or other oligonucleotide synthesizing enzymes.

The nucleic acid polymerizing enzyme utilized in accordance with the present invention can be either DNA polymerases, RNA polymerases, or reverse transcriptases. Suitable polymerases are thermostable polymerases or thermally degradable polymerases. Examples of suitable thermostable polymerases include polymerases isolated from Thermus aquaticus, Thermus thermophilus, Pyrococcus woesei, Pyrococcus furiosus, Thermococcus litoralis, and Thermotoga maritima. Useful thermodegradable polymersases include E. coli DNA polymerase, the Klenow fragment of E. coli DNA polymerase, T4 DNA polymerase, T7 DNA polymerase, and others. Examples for other polymerizing enzymes that can be used to determine the sequence of nucleic acid molecules include E. coli, T7, T3, SP6 RNA polymerases and AMV, M—MLV and HIV reverse transcriptases. The polymerase can be bound to the primed target nucleic acid sequence at a primed single-stranded nucleic acid, a double-stranded nucleic acid, an origin of replication, a nick or gap in a double-stranded nucleic acid, a secondary structure in a single-stranded nucleic acid, a binding site created by an accessory protein, or a primed single-stranded nucleic acid.

The oligonucleotide primer is present in the reagent composition in a molar excess concentration relative to the nucleotide analog concentrations. The oligonucleotide primer anneals to the target region of the PCR amplified genomic DNA template. Secondly, a nucleotide analog(s), catalyzed by DNA polymerase, extends the oligonucleotide primer by one nucleotide base complementary to the template immediately adjacent to the 3′ end of the primer thus consuming the nucleotide(s) from the reagent composition. The present invention provides for the identification of the nucleotide analog(s) that is consumed during the primer extension reaction by measuring the concentration of unreacted nucleotide analogs remaining in the reagent composition solution after primer extension.

In a preferred aspect of the present invention, after primer extension and before electrospraying, the extension solution is prepared for mass spectral analysis by first passing the reaction solution though a metal chelating resin, and then evaporating the effluent so that residual material is taken up in water. In order to maximize the amount of this residual material that dissolves in the water, the samples can be subjected to sonication. Sonication is carried out using a sonicator. Typically, sonication for a period of 5 to 10 minutes yields adequate sensitivity for mass spectral analysis.

Electrospray ionization provides for the atmospheric pressure ionization of a liquid sample (Kebaril et al., Electrospray Ionization Mass Spectrometry pp. 3-63 (1997), which is hereby incorporated by reference). The electrospray process creates highly-charged droplets that, under evaporation, create ions representative of the species contained in the solution. When a positive voltage is applied to the tip of the capillary relative to an extracting electrode, such as one provided at the ion-sampling orifice of a mass spectrometer, the electric field causes positively-charged ions in the fluid to migrate to the surface of the fluid at the tip of the capillary. If a negative voltage is applied to the tip of the capillary relative to an extracting electrode, such as one provided at the ion-sampling orifice to the mass spectrometer, the electric field causes negatively-charged ions in the fluid to migrate to the surface of the fluid at the tip of the capillary.

When the repulsion force of the solvated ions exceeds the surface tension of the fluid being electrosprayed, a volume of the fluid is pulled into the shape of a cone, known as a Taylor cone, which extends from the tip of the capillary. A liquid jet extends from the tip of the Taylor cone and becomes unstable and generates charged-droplets. These small charged droplets are drawn toward the extracting electrode. The small droplets are highly-charged and solvent evaporation from the droplets results in the excess charge in the droplet residing on the analyte molecules in the electrosprayed fluid. The charged molecules or ions are drawn through the ion-sampling orifice of the mass spectrometer for mass analysis. This phenomenon has been described, for example, by Dole et al., Chem. Phys. 49:2240 (1968) and Yamashita et al., J. Phys. Chem. 88:4451 (1984), which are hereby incorporated by reference. The potential voltage required to initiate an electrospray is dependent on the surface tension of the solution as described by, for example, Smith, IEEE Trans. Ind. Appl. IA-22:527-35 (1986), which is hereby incorporated by reference. Typically, the electric field is on the order of approximately 10⁶ V/m. The physical size of the capillary and the fluid surface tension determines the density of electric field lines necessary to initiate electrospray. Cole, Electrospray Ionization Mass Spectrometry: Fundamentals, Instrumentation, and Applications, (1997) summarizes much of the fundamental studies of electrospray. Several mathematical models have been generated to explain the principals governing electrospray.

U.S. patent application Ser. Nos. 09/468,535, 09/156,507, 60/176,605, and 60/210,890, as well as the application entitled “Multiple Electrospray Device, Systems, and Methods”, naming Gary A. Schultz, Thomas N. Corso, and Simon J. Prosser as inventors and filed Dec. 30, 2000 (Express Mail No. EL709323020US), which are hereby incorporated by reference, disclose suitable electrospray devices as well as methods and systems of using electrospray devices to prepare a sample for mass spectroscopy.

The electrospray device used in conjunction with the present invention includes a substrate having an injection surface and an ejection surface opposing the injection surface. The substrate is an integral monolith having one or more spray units for spraying the fluid. Each spray unit includes an entrance orifice on the injection surface, an exit orifice on the ejection surface, a channel extending between the entrance orifice and the exit orifice, and a recess surrounding the exit orifice and positioned between the injection surface and the ejection surface. The entrance orifices for each spray unit are in fluid communication with one another and each spray unit generates an electrospray of the fluid. The electrospray device also includes a first electrode attached to the substrate to impart a first potential to the substrate and a second electrode to impart a second potential. The first and the second electrodes are positioned to define an electric field surrounding the exit orifice.

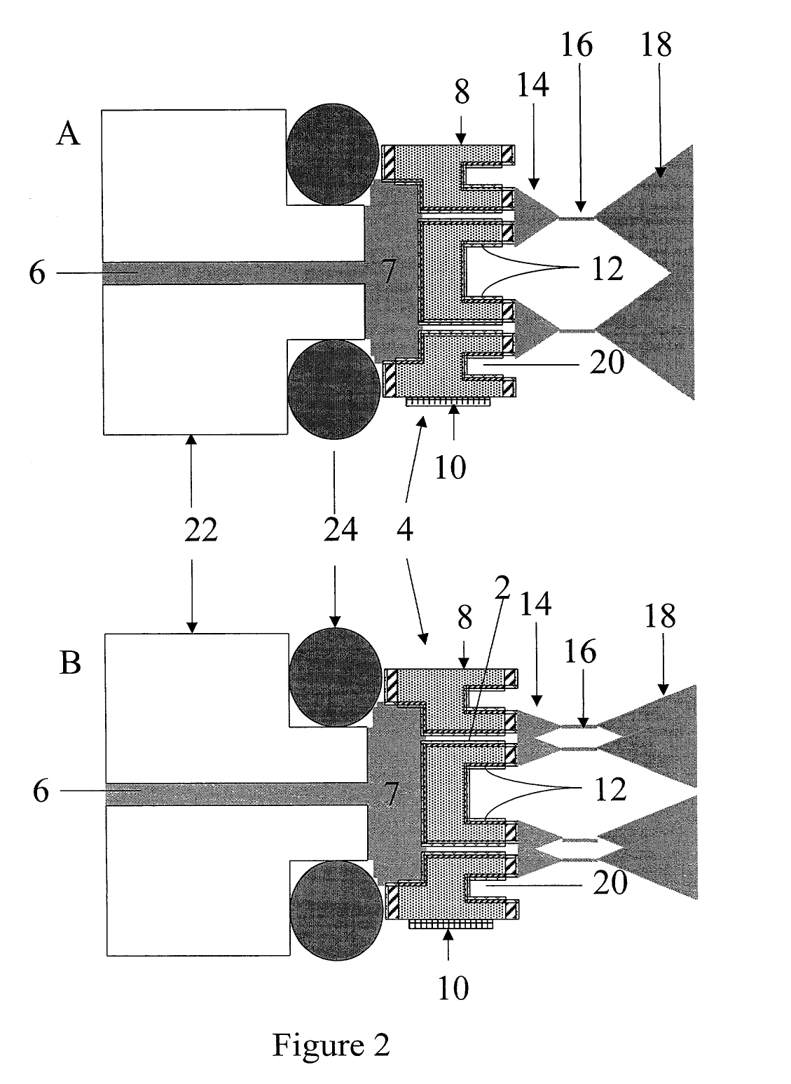

As shown in FIGS. 2A-B, to generate an electrospray, fluid may be delivered to the through-substrate channel 2 of the electrospray device 4 by, for example, a capillary 6, micropipette or microchip 22. Seal 24 is positioned between microchip 22 and electrospray device 4. The fluid is subjected to a potential voltage in the capillary 6 or in the reservoir 7 or via an electrode provided on the reservoir surface and isolated from the surrounding surface region and the substrate 8. A potential voltage may also be applied to the silicon substrate via the electrode 10 on the edge of the silicon substrate 8 the magnitude of which is preferably adjustable for optimization of the electrospray characteristics. The fluid flows through the channel 2 and exits from the nozzle 12 in the form of a Taylor cone 14, liquid jet 16, and very fine, highly charged fluidic droplets 18.

The nozzle 12 provides the physical asperity to promote the formation of a Taylor cone 14 and efficient electrospray 18 of a fluid. The nozzle 12 also forms a continuation of and serves as an exit orifice of the through-wafer channel 2. The recessed annular region 20 serves to physically isolate the nozzle 12 from the surface. The present invention allows the optimization of the electric field lines emanating from the fluid exiting the nozzle 12 through independent control of the potential voltage of the fluid and the potential voltage of the substrate 8.

The present invention also relates to a system that incorporates an array of reaction wells, preferably of volume less than 10 μL. The array is preferably in the same layout and spacing of standard 96, 384, 1536, and 6,144 well plates, although any array is suitable and may be optimized for a given application. The reaction wells contain a buffering solution, magnesium acetate, DNA polymerase, amplified target DNA, and SNP primer in a molar excess relative to the concentrations of the four ddNTPs (ddCTP, ddTTP, ddATP, and ddGTP) for performing SNP primer extension reactions followed by quantification of free ddNTPs remaining in each reaction well.

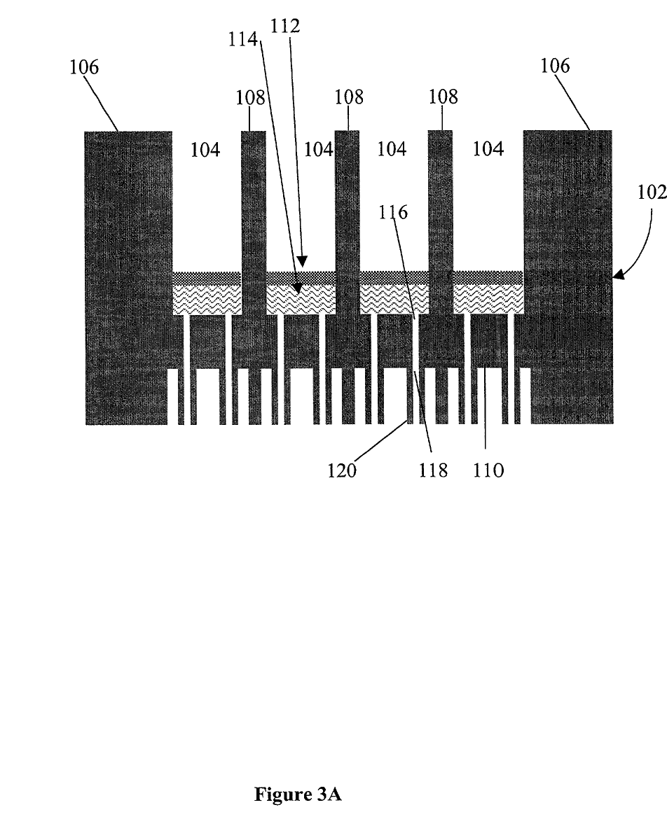

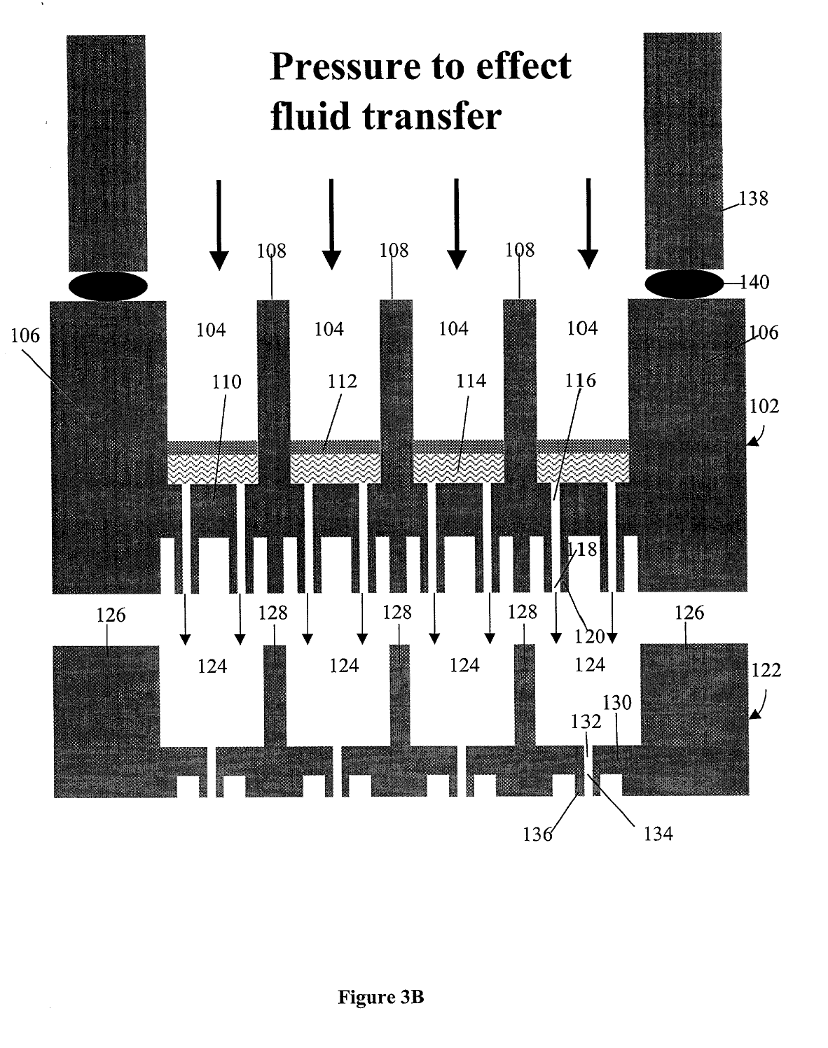

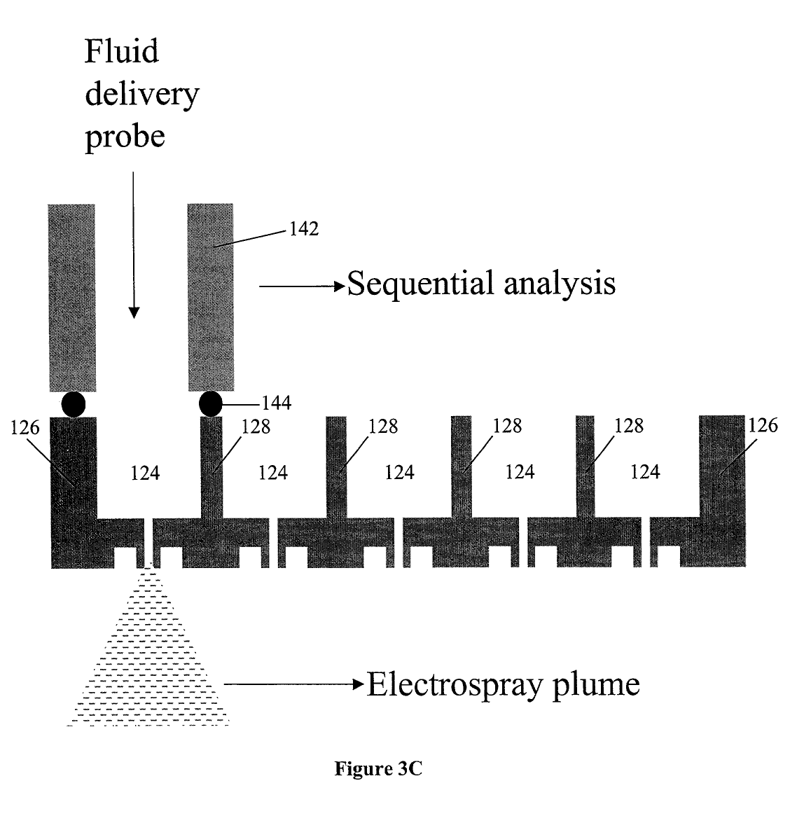

Another aspect of the present invention relates to a reaction well block for performing a reaction, such as polymerase chain reaction and primer extension. As shown in FIG. 3A, this aspect of the present invention is in the form of an array 102 of reaction wells 104 formed between plate edges 106 and/of walls 108. Wells 104, proximate to base 110, contain frit 112 or other medium separating the solution from the metal chelating resin. Liquid is discharged from wells 104 into entrance orifice 116, through channel 118, and out of exit orifice 120.

The system incorporates reaction wells with volumes on the order of tens of microliters to less than a microliter. The present invention has several advantages over other systems disclosed in the prior art. The double-stranded amplified target DNA fragment can be added directly to the reaction well array without prior separation of the strands. The SNP primers can be free in solution, thus increasing the reaction probability with the target DNA during the primer extension thermal cycles. The SNP primer used for each reaction is also an excess reagent relative to the added amount of each of the ddNTPs, thus effectively improving the incorporation efficiency (rate) of the target dideoxynucleotide base(s). The ddNTPs are added as a limiting reagent so that the ddNTPs that react and extend the SNP primer will be substantially consumed from the reaction solution. The reaction solution is then passed through a metal chelating resin either on- or off-line to prepare the solution for electrospray mass spectrometry analysis. The relative response of the four ddNTP bases identifies by which base(s) the SNP primer was extended. Heterozygous SNPs can be identified if two ddNTP bases react with the SNP primer. In addition, this method can be used for discovery of the known point variation with both tri-allelic and tetra-allelic SNPs.

Another aspect of the present invention relates to an electrospray system. This system includes an electrospray device which comprises a substrate having an injection surface and an ejection surface opposing the injection surface. The substrate is an integral monolith having an entrance orifice on the injection surface, an exit orifice on the ejection surface, a channel extending between the entrance orifice and the exit orifice, and a recess extending into the ejection surface and surrounding the exit orifice to define a nozzle on the ejection surface. The electrospray system also includes a sample preparation device, as shown in FIG. 3A, positioned to transfer fluids to the electrospray device where the sample preparation device comprises a liquid passage and a metal chelating resin positioned to treat fluids passing through the liquid passage. Instead of a metal chelating agent, the sample preparation device can have a molecular weight filter positioned to treat fluids passing through the liquid passage.

This electrospray system is shown in FIG. 3B and includes array 102 of reaction wells 104 each positioned to discharge liquid into electrospray microchip 122. In particular, each exit orifice 120 is positioned to discharge liquid into a particular receiving well 124 which is formed between edges 126 and/or walls 128. After making this transfer, solutions evaporate in receiving wells 124 to dryness and are subsequently hydrated for controlled discharge. Liquid is discharged from receiving well 124 through base 130 via entrance orifice 132, channel 134, and exit orifice 136. As a result, liquid is discharge from electrospray microchip 122 as an electrospray. Preferably, electrospray microchip 122 is positioned in front of an ion-sampling orifice of an atmospheric pressure ionization mass spectrometer for analysis of the ddNTPs.

Another preferred embodiment would interface a microchip-based array of separation channels for the detection of ddNTPs with the reaction well array. The ddNTPs may be separated by liquid chromatography or electrophoretic methods and quantified using spectroscopic or conductometric detection. A multi-system chip can be fabricated using Micro-ElectroMechanical System (MEMS) technology (Schultz et al., Anal Chem 72: 4058-63 (2000), which is hereby incorporated by reference) to further provide a rapid sequential chemical analysis system for large-scale SNP genotyping. For example, the multi-system chip enables automated, sequential separation and injection of a multiplicity of samples, resulting in significantly greater analysis throughput and utilization of the mass spectrometer instrument for high-throughput SNP detection.

As shown in FIG. 3B, liquid is fed into the entire depicted array 102 of reaction wells 104 through conduit 132. A seal 140 is positioned between edge 106 and conduit 138 to prevent leakage. In addition, as shown FIG. 3C, a fluid delivery probe 142 is positioned against edges 126 and/or walls 128 by means of seal 144 to permit liquid to be charged to the individual receiving wells 124. After each receiving well is filled, probe 142 can move sequentially to the next well and fill it.

In a preferred embodiment, the present invention is performed using an array of reaction wells. The array of reaction wells is multi-layered. The top layer consists of a reaction well. The middle layer has a sample cleanup phase, preferably a metal chelating resin, for the removal of magnesium from the reaction mixture. Also, a frit and a molecular weight filter may be used. The bottom layer has receiving wells in fluid communication with nozzles contained on a microchip for generating an electrospray of the reaction well product solution.

Due to its sensitivity and specificity with regard to low molecular weight entities, mass spectrometry is preferably used for the detection of these four ddNTPs independent of the SNP under evaluation. The mass spectrometry instrument and detection method is setup to screen any SNP by monitoring four unique ion response channels, one for each ddNTP. By use of nanomolar detection sensitivity, the electrospray mass spectrometry method is able to provide a rapid, selective, and sensitive method for SNP screening.

A further aspect of the present invention is directed to a reagent composition which includes an aqueous carrier, an oligonucleotide primer, a mixture of nucleotide analogs of different types, magnesium acetate, a buffer, and a nucleic acid polymerizing enzyme. According to this embodiment of the present invention, there can be an excess of the oligonucleotide primer to nucleotide analog or there is a limited concentration of nucleotide analogs present in the composition. The buffer can be ammonium bicarbonate, ammonium acetate buffer, or mixtures thereof. Suitable ranges of these components in the composition are 1-150 nM of PCR product, 1-10 μM of SNP primer, 0.1-10 μM each of the ddATP, ddCTP, ddGTP, and ddTTP nucleotide analogs, 1-50 mM NH₄Ac buffer at a pH of 8.7, 0.5-4 mM Mg(Ac)₂, and 0.1-5 unit of DNA polymerase. Preferred amounts of the components are 50 nM of PCR product, 4 μM of SNP primer, 1 μM each of the ddATP, ddCTP, ddGTP, and ddTTP nucleotide analogs, 20 mM NH₄Ac buffer at a pH of 8.7, 2 mM Mg(Ac)₂, and 1 unit of DNA polymerase.

### EXAMPLES

### Example 1—Mass Spectral Analyses

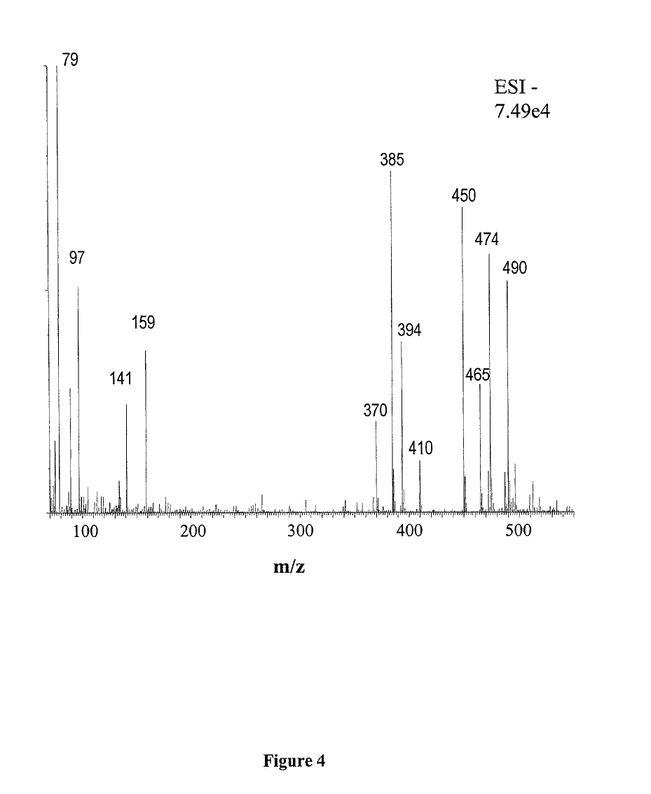

By continuously infusing 10 μM ddNTPs at a rate of 10 μL/min into a stream of mobile phase flowing at 50 μL/min, electrospray mass spectra of the ddNTPs were determined. The cone voltage was 25 V, and the desolvation temperature was 400° C. The mobile phase consisted of 50/50 methanol/water with 0.1% acetic acid. In the mass spectrum, the pseudomolecular ions, (M—H)⁻, of ddCTP, ddTTP, ddATP, and ddGTP appeared at m/z 450, m/z 465, m/z 474, and m/z 490, respectively, as shown in FIG. 4. In addition, the (M—PO₃H₂)⁻ ions for each of the bases, ddCTP, ddTTP, ddATP, and ddGTP, formed by fragmentation in the source of the mass spectrometer were observed. Other ions were observed at m/z 79, corresponding to PO₃⁻, and m/z 159, corresponding to HP₂O₆⁻.

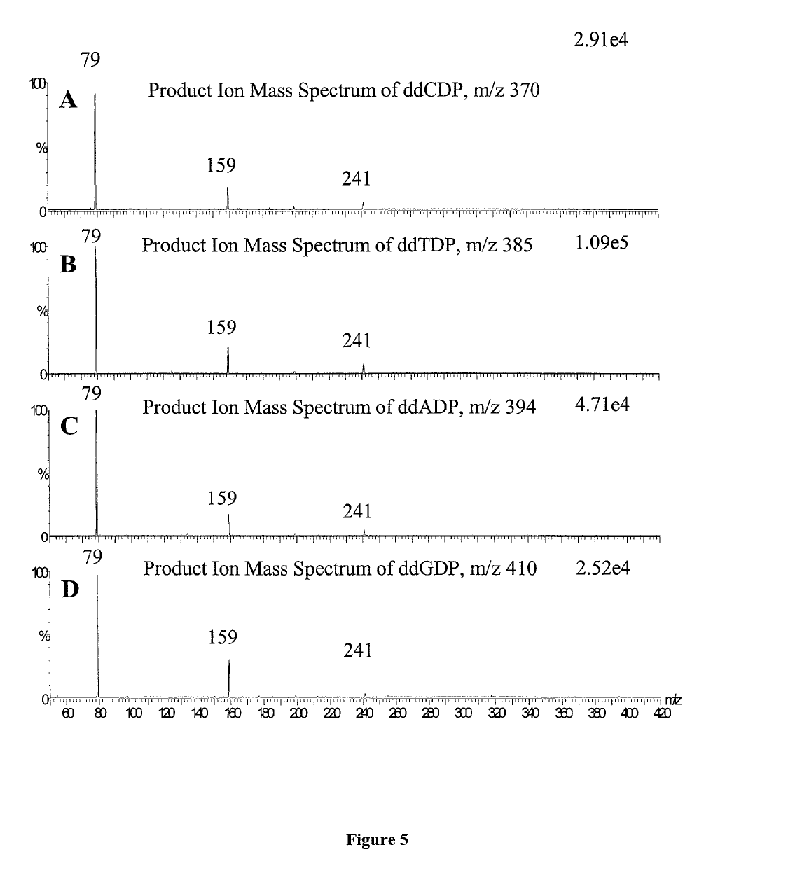

The MS/MS product ion mass spectra of the (M—PO₃H₂)⁻ ions for each of the four ddNTPs was obtained by continuously infusing 10 μM ddNTPs at a rate of 10 μL/min into a stream of mobile phase flowing at 50 μL/min. The mobile phase consisted of 0.1% acetic acid. The (M—PO₃H₂)⁻ ions were isolated and then collisionally dissociated using a collision energy of 35 eV. The cone voltage and desolvation temperature were maintained at 25 V and 400° C., respectively. The mass spectrometer was scanned over the range of 50 m/z to 420 m/z, detecting the product ions formed. As shown in FIGS. 5A-D, product ions were observed at m/z 79, 159 and 241 for all four bases.

Selected reaction monitoring (SRM) is an experiment where the mass spectrometer is set up to acquire data for a unique precursor ion to product ion transition for mixtures of analytes. This SRM experiment allows for unique signals to be obtained on analytes contained in complex mixtures without interference from other compounds contained within the mixture. In practice, this firstly involves the isolation of a precursor ion in one region of the mass spectrometer, secondly, focusing that ion into a collision cell to cause the ion to fragment and form product ions that are related to the molecular structure of the precursor ion. Thirdly, focusing the product ions into another region of the mass spectrometer and mass selecting one of the product ions formed in the collision cell for detection.

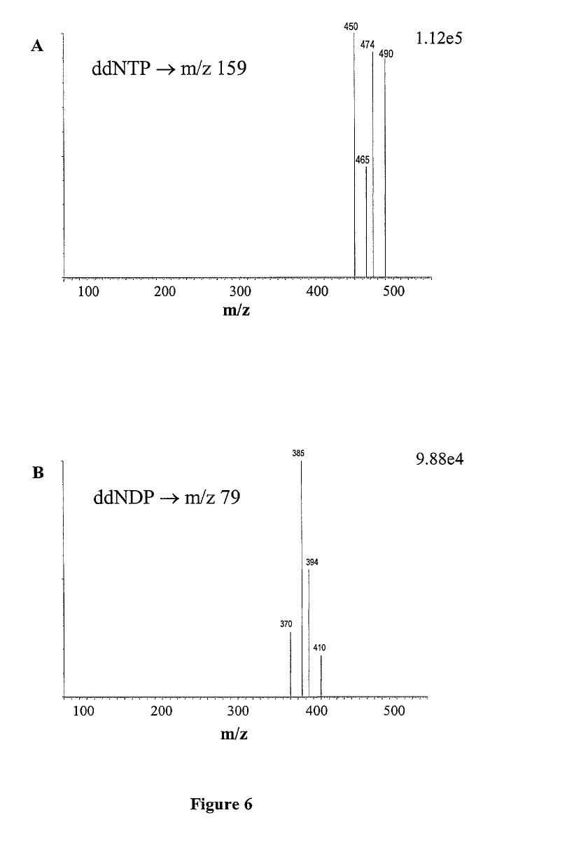

SRM MS/MS mass spectra for the (M—H)⁻ ions collisionally dissociated to the common product ion m/z 159 and for the (M—H₂PO₃)⁻ ions collisionally dissociated to the common product ion m/z 79, respectively, were obtained by continuously infusing 10 μM ddNTPs at a rate of 10 μL/min into a stream of mobile phase flowing at 50 μL/min. The mobile phase consisted of 50/50 methanol/water with 0.1% acetic acid. The (M—H)⁻ and (M—H₂PO₃)⁻ ions for each of the four ddNTPs was first isolated and then collisionally dissociated. The product ion m/z 159 or m/z 79, common to all four bases, were monitored. The dwell time for each transition was 200 msec, the collision energy was 25 eV for (M—H)⁻ and 35 eV for (M—H₂PO₃)⁻, the cone voltage was 25 V, and the desolvation temperature was maintained at 400° C. For the (M—H)⁻ ions, the SRM transitions monitored were as follows: ddCTP, m/z 450.1→m/z 159.0; ddTTP, m/z 465.1→m/z 159.0; ddATP, m/z 474.1→m/z 159.0; ddGTP, m/z 490.1→m/z 159.0. See FIG. 6A. For the (M—H₂PO₃)⁻ ions, the SRM transitions monitored were as follows: ddCTP, m/z 370.1→m/z 79.0; ddTTP m/z 385.1→m/z 79.0; ddATP, m/z 394.1→m/z 79.0; ddGTP, m/z 410.1→m/z 79.0. See FIG. 6B. The ion abundance for each transition was represented by the precursor ion, because the product ion m/z 79 and m/z 159 is common to all four bases.

### Example 2—Effect of Magnesium Removal

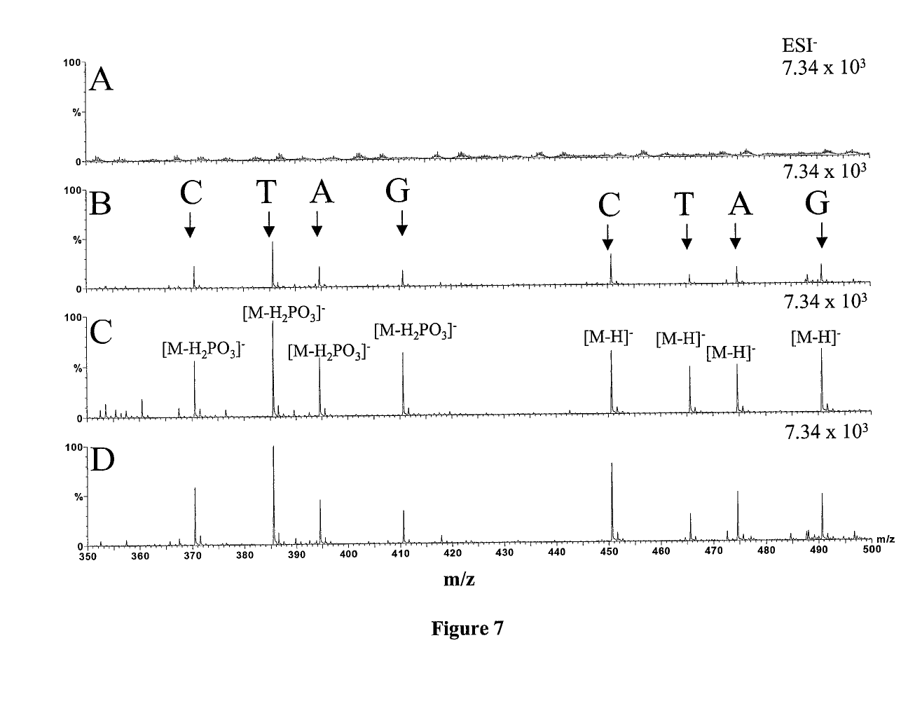

Well product solutions were evaporated to dryness and reconstituted in a 0.01% acetic acid in methanol solution to demonstrate the importance of removing magnesium prior to electrospray mass spectrometry of the reaction well product solutions. FIG. 7A shows the mass spectrum of a solution of 1 μM ddNTPs (C, T, A, G) in 20 mM ammonium acetate pH 8.7, 1 mM magnesium acetate. Note the absence of a signal in the mass spectrum for each of the ddNTPs. FIG. 7B shows the mass spectrum of this same solution passed through a metal chelating resin based on iminodiacetic acid (IDA) functional groups used to complex with metals including magnesium. The metal chelating resin removes the magnesium from the solution resulting in a measurable signal for each of the ddNTPs as labeled in the mass spectrum. FIG. 7C shows the mass spectrum of a solution of 1 μM ddNTPs (C, T, A, G) in 20 mM ammonium acetate pH 8.7 without magnesium acetate and also that was passed through the metal chelating resin. FIG. 7D shows the mass spectrum of a solution of 1 μM ddNTPs (C, T, A, G) in 20 mM ammonium acetate pH 8.7 that has only been evaporated to dryness and reconstituted prior to electrospray mass spectrometry analysis. Note that there is no difference between the relative ion intensities for the four ddNTPs of the control experiment in FIG. 7D to that in FIG. 7C, indicating that the IDA metal chelating resin does not adversely adsorb the ddNTPs. In FIG. 7B, with the presence of magnesium, the measured signals were reduced to approximately one-half the control shown in FIG. 7D. In the case of FIG. 7A, where high levels of magnesium were present in the solution, the formation of ddNTP ions using electrospray was markedly reduced.

### Example 3—Optimization of Primer Extension Reaction

To simplify the primer extension reaction, a synthetic oligonucleotide, template A, (5′ CCCCTGTATCCTGTGTGAAATTGTTATCCGCTC 3′ (SEQ. ID. No. 1) 33mer) corresponding to the flanking region of the poly-restriction sites of pUC18/19 plasmid, was used as a target template. A universal primer #1233 (5′ AGCGGATAACAATTTCACACAGGA 3′ (SEQ. ID. No. 5) 24mer) which is a complement to the above synthetic template, was used as the SNP primer. The reaction was set up in a total volume of 50 μL with 25 mM ammonium acetate buffer pH 9.3, 1 μM ddNTPs, 2 mM magnesium acetate, 0.1 μM template A, and 1 unit of Thermoequenase (Amersham). The #1233 primer was varied at concentrations of 0 μM, 1 μM, 2 μM, 3 μM, and 4 μM in the reaction for a total of five samples. The reaction mixture was subjected to 25 thermal cycles in a GeneAmp PCR System 9700 (PE Biosystem) with each cycle consisting of 95° C. for 30 sec, 60° C. for 60 sec, and 72° C. for 60 sec. The extended reaction samples were passed through Ultrafree-0.5 filter units (Millipore) and a micro metal chelating column composed of immobilized iminodiacetic acid gel (Pierce). The resulting samples were analyzed by electrospray ionization coupled to a triple quadrupole Quattro II (Micromass) mass spectrometer (ESI-MS/MS). A mobile phase composition of 1:1 methanol:water with 0.1% acetic acid was used at a flow rate of 150 μL/min. At least three 10 μL injections were made for each sample via loop injection into the mobile phase. The mass spectrometer was operated in MS/MS selected reaction monitoring (SRM) mode for each base. The following SRM transitions were monitored for each of the bases: ddCTP, m/z 370.1→m/z 79.0; ddTTP, m/z 385.1→m/z 79.0; ddATP, m/z 394.1→m/z 79.0; ddGTP, m/z 410.1→m/z 79.0.

### Example 4—Determination of Suitable Primer Concentration

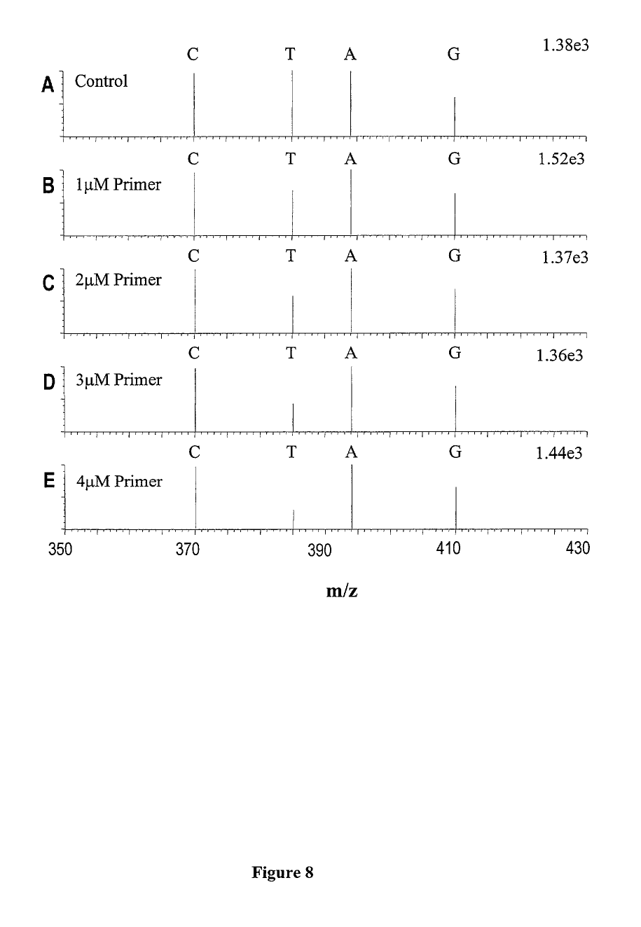

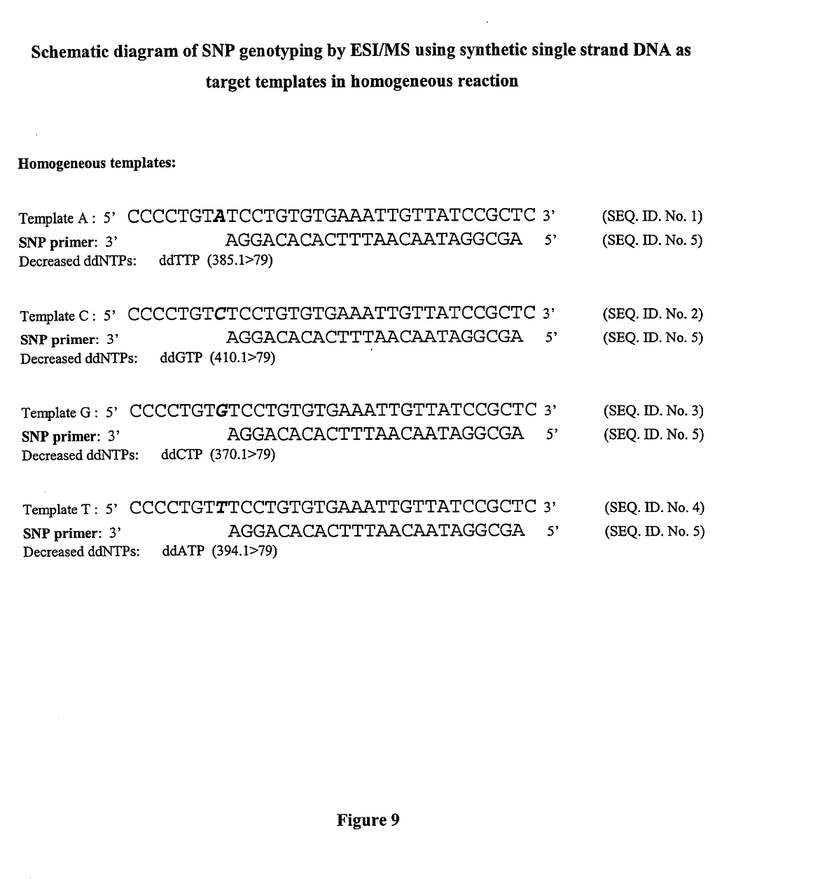

To determine what concentration of primer should be used, an ESI-MS/MS spectra for the above five samples was determined. In these experiments, the extension reaction mixtures each contained 1 μM ddNTPs, 2.5 units of Thermosequenase (Amersham), 2 mM magnesium acetate, 25 mM ammonium acetate pH 9.3, 0.1 μM template A (sequence shown in FIG. 9), and varying concentrations of SNP primer (sequence shown in FIG. 9). The concentrations of primer in the reactions for FIGS. 8B, C, D, and E, were 1 μM, 2 μM, 3 μM, and 4 μM, respectively. The control reaction, shown in FIG. 8A, was identical to the reaction FIG. 8D, except that the Thermosequenase was omitted. The primer extension reaction consisted of 25 cycles with each cycle composed of a 30 sec denaturing step at 95° C., a 60 sec annealing step at 60° C., and a 60 sec extension step at 72° C. The extension reaction samples were prepared by filtering with an Ultrafree—0.5 micron filter unit followed by solid phase extraction using an immobilized iminodiacetic acid gel column. With template A, the SNP base was A. Therefore, following the extension reaction, it was expected that the concentration of ddTTP, which corresponds to the transition m/z 385.1→m/z 79.0, would decrease due to its incorporation at the 3′ end of the primer. The mass spectral data showed that as the primer concentration increased, the consumption of ddTTP in the extension reaction also increased, resulting in a decrease in the ion abundance of transition m/z 3 85.1→m/z 79.0. These data reveal that the optimal primer concentration is 4 μM in the primer extension reaction. The relative peak area ratios of the various transitions are displayed in Table 1.

TABLE 1
Summary of the Peak Area Ratios of PCR Extension Reaction Samples
which Contained Varying Primer Concentrations.

| | Peak Area Ratios | | | | | |
|---|---|---|---|---|---|---|
| Sample | 370/385 | 370/394 | 370/410 | 385/394 | 385/410 | 394/410 |
| Control | 0.94 | 0.95 | 1.93 | 1.01 | 2.05 | 2.03 |
| 1 μM Primer | 1.59 | 0.89 | 1.83 | 0.56 | 1.16 | 2.05 |
| 2 μM Primer | 2.48 | 0.97 | 1.69 | 0.39 | 0.68 | 1.74 |
| 3 μM Primer | 5.40 | 0.90 | 1.58 | 0.17 | 0.30 | 1.75 |
| 4 μM Primer | 6.73 | 0.94 | 1.65 | 0.14 | 0.25 | 1.76 |

Note:
370 denotes the transition m/z 370.1 → m/z 79.0
385 denotes the transition m/z 385.1 → m/z 79.0
394 denotes the transition m/z 394.1 → m/z 79.0
410 denotes the transition m/z 410.1 → m/z 79.0

By mathematically adjusting the relative ratios of the bases for all reactions, it is estimated that up to 86% of the initial ddTTP reacted to extend the primer in the sample containing 4 μM SNP primer.

### Example 5—SNP Assay Using Synthetic Oligonucleotides as Homozygous Templates

To determine the utility of this SNP assay, a model system was adopted with one SNP primer (#1233 primer) (SEQ. ID. No. 5) and four synthetic 33-mer templates (SEQ. ID. Nos. 1-4). These four templates differed by only one SNP base, A, C, G, or T, as shown in FIG. 9. To detect all possible SNP base alterations for homozygous cases, 0.1 μM of each of the four templates were used for SNP extension reactions under aforementioned conditions. A universal reverse primer #1233 (BioLabs) was used for extension. The polymorphic site (A) at position 8 is shown in bold and italics in template A. Other targets including template C, template G, and template T were identical to template A except for a C, G, or T at position 8, respectively. The ddNTP expected to be consumed in the primer extension reaction for each template is indicated. FIG. 9 shows the results of SNP genotyping by ESI-MS/MS using synthetic single-stranded DNA as target templates. All reactions, including control samples that did not contain template were run in duplicate.

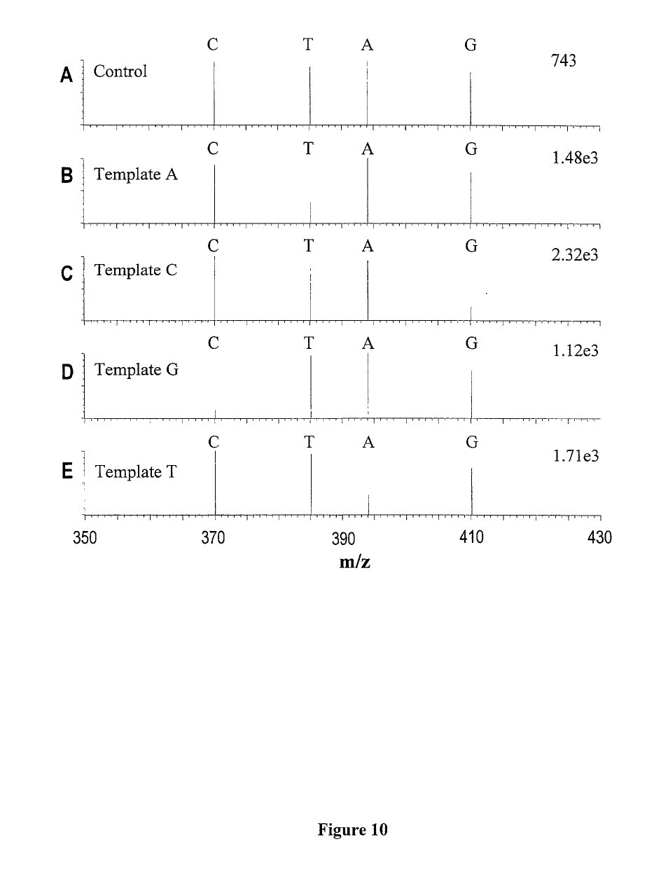

To ensure that the technique of the present invention would correctly identify the four possible SNP bases, A, C, G, and T, four different templates whose sequences are shown in FIG. 9, were synthesized. These templates differed from one another only by one base at position 8 and were named by this polymorphic base, so that the same primer could be used in the extension reaction for all four templates. The extension reaction mixtures each contained 1 μM ddNTPs, 1.25 units of Thermosequenase, 2 mM magnesium acetate, 25 mM ammonium acetate pH 9.3, 0.2 μM template, and 4 μM primer. These reactions differed from one another only by the particular template used in each. The control reaction in FIG. 10A was identical to the others except that it did not contain template. The extension reaction was thermally cycled for 25 cycles with each cycle composed of a 30 sec denaturing step at 95° C., a 60 sec annealing step at 60° C., and a 60 sec extension step at 72° C. The extension reaction samples were prepared for mass spectral analysis by filtering with an Ultrafree—0.5 micron filter unit followed by solid phase extraction using an immobilized iminodiacetic acid gel column. The reaction in FIG. 10B contained template A which has the SNP base A. Therefore, during the extension reaction, it was expected that ddTTP, corresponding to the transition m/z 385.1→m/z 79.0, would be incorporated into the primer. The resulting decrease in intensity of the m/z 385.1→m/z 79.0 transition is shown in the reaction of FIG. 10B. In the reaction of FIG. 10C, template C having the SNP base C, was used. Here, it was expected that following the extension reaction, ddGTP, corresponding to the transition m/z 410.1→m/z 79.0. would be consumed. This was observed in the reaction of FIG. 10C, with a significant decrease in the ion intensity of ddGTP, m/z 410.1→m/z 79.0. Template G, with SNP base G, was used in extension reaction FIG. 10D. A decrease in ddCTP, corresponding to the transition m/z 370.1→m/z 79.0, was expected and observed. Finally, the last possible SNP base T, in template T was used in the reaction of FIG. 10E. Here, it was expected that ddATP, m/z 394.1→m/z 79.0, would be incorporated into the primer. A decrease in the ion intensity of m/z 394.1→m/z 79.0 was observed in the reaction of FIG. 10E. These results show that the analysis of the present invention can unambiguously identify the four possible bases. The relative peak area ratios of the various transitions are displayed in Table 2.

TABLE 2
Summary of the Peak Area Ratios of PCR Extension Reaction Samples
Containing Homogeneous Single-Stranded DNA Template.
The four templates used were
named by their polymorphic base. Samples were prepared in duplicate.

| | Peak Area Ratios | | | | | |
|---|---|---|---|---|---|---|
| Sample | 370/385 | 370/394 | 370/410 | 385/394 | 385/410 | 394/410 |
| Control | 1.31 | 0.97 | 1.40 | 0.86 | 1.24 | 1.44 |
| Control | 1,03 | 0.98 | 1.38 | 0.95 | 1.33 | 1.40 |
| Template A | 11.96 | 0.93 | 1.26 | 0.08 | 0.11 | 1.35 |
| Template A | 12.93 | 1.07 | 1.50 | 0.08 | 0.12 | 1,41 |
| Template C | 1.24 | 1.05 | 10.20 | 0.85 | 8.24 | 9.64 |
| Template C | 1.23 | 1.07 | 9.77 | 0.87 | 7.96 | 9.18 |
| Template G | 0.17 | 0.16 | 0.23 | 0.93 | 1,36 | 1.46 |
| Template G | 0.19 | 0.17 | 0.26 | 0.89 | 1.34 | 1.50 |
| Template T | 1.05 | 7.62 | 1.52 | 7.24 | 1.45 | 0.20 |
| Template T | 1.05 | 7.20 | 1.43 | 6.87 | 1.36 | 0.20 |

Note:
370 denotes the transition m/z 370.1 → m/z 79.0
385 denotes the transition m/z 385.1 → m/z 79.0
394 denotes the transition m/z 394.1 → m/z 79.0
410 denotes the transition m/z 410.1 → m/z 79.0

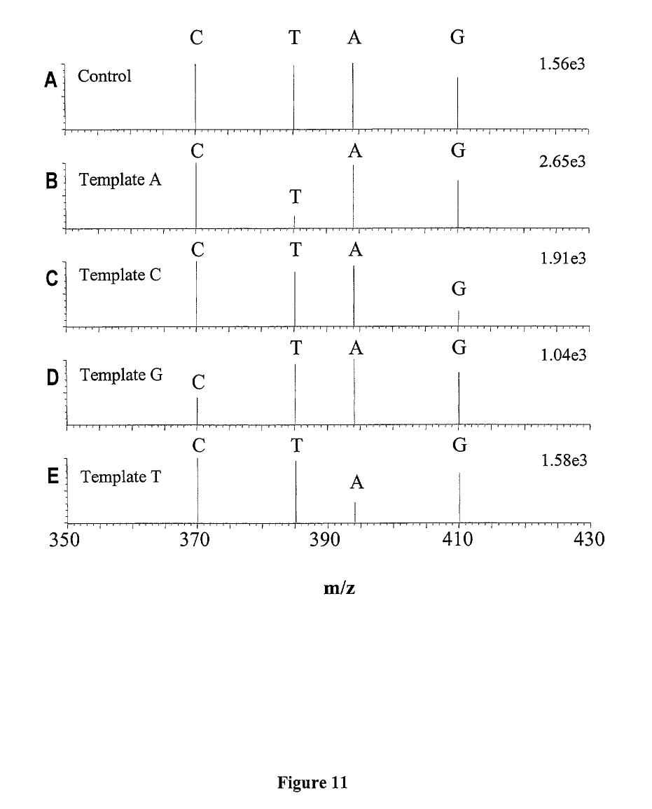

FIG. 11 shows the results from the duplicate set of samples. Both FIGS. 10 and 11 show identical results with the expected bases consumed by 70-80% of their initial concentration. Therefore, this method of SNP analysis provides unambiguous identification of all possible single (homozygous) SNP bases.

### Example 6—SNP Assay Using Synthetic Oligonucleotides as Heterozygous Templates

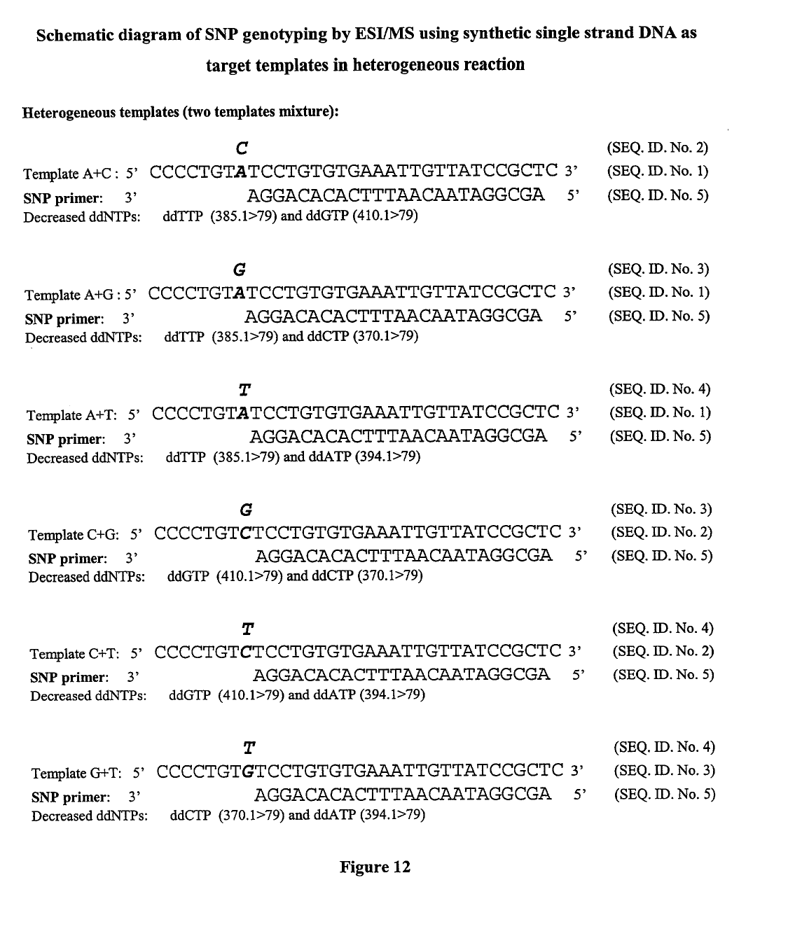

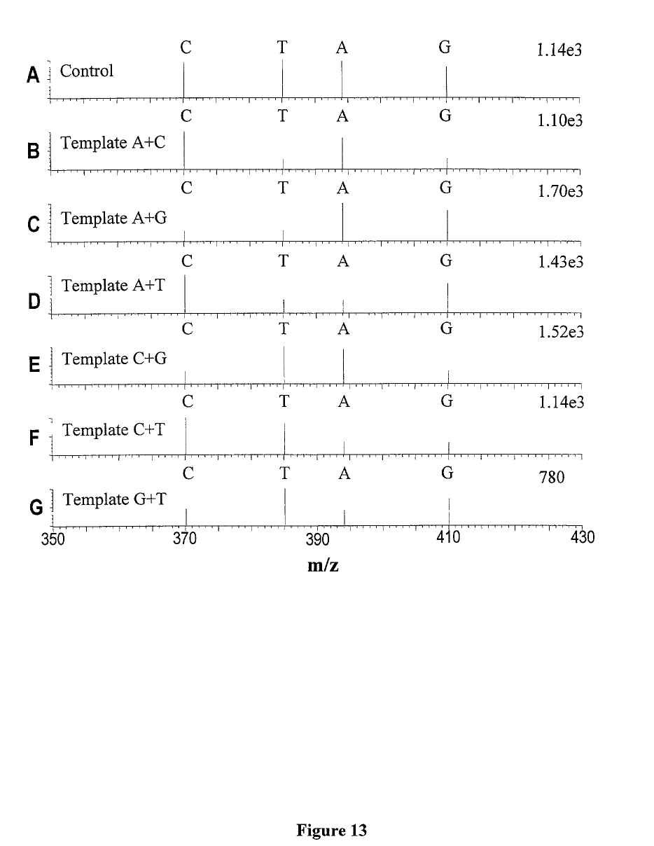

To mimic the double SNP base changes in heterozygous cases, mixtures of the 33-mer templates (SEQ. ID. Nos. 1-4) outlined in FIG. 9 were combined at a concentration of 0.025 μM for each of two templates in the SNP extension reactions with the SNP primer (SEQ. ID. No. 5), as shown in FIG. 12. The results from duplicate sample preparations for the heterozygous cases are shown in FIG. 13. These samples represent heterozygous cases where two polymorphic bases are simultaneously present. These samples represent all possible heterozygous possibilities. The equal molar mixture of two single-stranded DNA templates, named by the SNP bases that were used in previous experiment were also used here. The extension reaction mixtures each contained 1 μM ddNTPs, 1.25 units of Thermosequenase, 2 mM magnesium acetate, 25 mM ammonium acetate pH 9.3, 4 μM primer, and 0.1 μM each of two different templates. The particular templates used in each reaction are provided in FIG. 12. The control reaction was identical to the others except that it did not contain any template. The extension reaction was thermally cycled for 25 cycles with each cycle composed of a 30 sec denaturing step at 95° C., a 60 sec annealing step at 60° C., and a 60 sec extension step at 72° C. The extension reactions samples were prepared for mass spectral analysis by filtering with an Ultrafree—0.5 micron filter unit followed by solid phase extraction using an immobilized iminodiacetic acid gel column. When comparing the reaction of FIG. 13B to the reaction of FIG. 13A, it is apparent that ddTTP, corresponding to the transition m/z 385.1→m/z 79.0, and ddGTP, corresponding to the transition m/z 410.1→m/z 79.0, have decreased in intensity. This is consistent with what would be expected when polymorphic bases A and C are present as they were in the reaction of FIG. 13B. Templates A and G were present in the reaction of FIG. 13C, and, as expected ddTTP, m/z 385.1→m/z 79.0, and ddCTP, m/z 370.1→m/z 79.0, decreased in ion intensity. In the reaction of FIG. 13D, the SNP bases are A and T, and the corresponding ddTTP, m/z 385.1→m/z 79.0, and ddATP, m/z 394.1→m/z 79.0, were observed to decrease in intensity. The presence of templates C and G in the reaction of FIG. 13E, resulted in the expected decrease in ion abundance of ddGTP, m/z 410.1→m/z 79.0, and ddCTP, m/z 370.1→m/z 79.0. In the reaction of FIG. 13F, ddATP, m/z 394.1→m/z 79.0, and ddGTP, m/z 410.1→m/z 79.0, decreased in intensity, corresponding to the expected consumption of ddATP and ddGTP in the primer extension reaction in the presence of polymorphic bases T and C. In the reaction of FIG. 13G, templates G and T, corresponding to polymorphic bases, G and T, the expected decrease in ion abundance of ddCTP, m/z 370.1→m/z 79.0, and ddATP, m/z 394.1→m/z 79.0 was achieved. This experiment shows that this analysis technique can be used to determine the polymorphic bases in heterozygous cases. In each sample, both of the bases expected to decrease in concentration did in fact decrease, with each base consumed by 70-80% of its initial concentration despite the fact that only half the amount of each template was added. This result reveals that all possible combinations for heterozygous polymorphisms can be easily identified by the method of the present invention and, in addition, that the 25 thermal cycles used for the extension reaction are in kinetic excess for efficient incorporation of the free ddNTPs. Table 3 lists the peak area ratios for all duplicate samples in the heterogeneous reactions.

TABLE 3
Summary of the Peak Area Ratios of PCR Extension Reaction Samples
Containing Heterogeneous Single-Stranded DNA Templates.
This data mimics heterozygous cases. The four templates
used were named by their polymorphic base.
Samples were prepared in duplicate.

Peak Area Ratios

| | | | | | | 394/ |
|---|---|---|---|---|---|---|
| Sample | 370/385 | 370/394 | 370/410 | 385/394 | 385/410 | 410 |
| Control | 0.92 | 0.97 | 1.28 | 1.06 | 1.40 | 1.32 |
| Control | 0.80 | 0.97 | 1.19 | 1.22 | 1.49 | 1.23 |
| Template A + C | 8.52 | 1.27 | 8.79 | 0.15 | 1.04 | 6.94 |
| Template A + C | 9.06 | 1.22 | 8.63 | 0.14 | 0.98 | 7.08 |
| Template A + G | 1.30 | 0.15 | 0.21 | 0.12 | 0.16 | 1.38 |
| Template A + G | 1.11 | 0.15 | 0.21 | 0.14 | 0.19 | 1.37 |
| Template A + T | 7.36 | 5.52 | 1.46 | 0.75 | 0.20 | 0.27 |
| Template A + T | 7.01 | 5.98 | 1.70 | 0.90 | 0.25 | 0.29 |
| Template C + C | 0.16 | 0.18 | 1.13 | 1.09 | 6.89 | 6.32 |
| Template C + G | 0.11 | 0.13 | 0.85 | 1.14 | 7.77 | 6.78 |
| Template C + T | 1.20 | 6.15 | 6.63 | 5.16 | 5.54 | 1.10 |
| Template C + T | 1.31 | 6.95 | 9.52 | 5.30 | 7.29 | 1.40 |
| Template G + T | 0.17 | 1.44 | 0.30 | 8.35 | 1.73 | 0.21 |
| Template G + T | 0.17 | 1.17 | 0.26 | 6.74 | 1.52 | 0.23 |

Note:
370 denotes the transition m/z 370.1 → m/z 79.0
385 denotes the transition m/z 385.1 → m/z 79.0
394 denotes the transition m/z 394.1 → m/z 79.0
410 denotes the transition m/z 410.1 → m/z 79.0

Using the peak area ratios for all combinations of the four oligonucleotide bases allows for the detection of changes in the relative concentrations of the bases. Through data analysis, the nature of the SNP locus is readily determined as either a homozygous or heterozygous polymorphism. Furthermore, the relative standard deviation of the peak area ratio data for each sample and its duplicate, encompassing six injections was typically less than 15%, suggesting this method of genotyping SNPs by detecting free ddNTPs is reproducible.

### Example 7—SNP Assay Using Amplified Double-Stranded DNA as Template

The model system described previously consisted of a single-stranded DNA target sequence. However, from a practical standpoint, double-stranded DNA will be encountered more often. A potential problem for using double-stranded DNA is the reannealing of the two complementary strands that could compete with the SNP primer and thereby lower the rate of the extension reaction. To determine whether the method of the present invention is applicable to double-stranded DNA, amplified double-stranded DNA was used as the template in a primer extension reaction. An E. coli PheA gene was cloned in pUC18 to make a pJS1 plasmid (Zhang et al., J Biol Chem 273: 6248-53 (1998), which is hereby incorporated by reference). A 384 bp portion of partial E. coli PheA gene (SEQ. ID. No. 6) was amplified by regular PCR using this pJS1 as a template along with W338Ipd (SEQ. ID. No. 7) as the forward primer and #1224 (SEQ. ID. No. 8) as the reverse primer. The PCR amplification utilized AmpliTaq DNA polymerase and a GeneAmp PCR System 9700 (PE Biosystem). The amplification was performed in 35 thermal cycles with each cycle consisting of 95° C. for 30 sec, 60° C. for 60 sec, and 72° C. for 60 sec. The resulting PCR product was passed through a Microcon-50 filter unit (Millipore) to isolate the 384 bp template from the residual free dNTPs and primers. The concentrated 384 bp PCR product was then quantified spectrophotometrically (OD260 nm) and used for the following extension reaction.

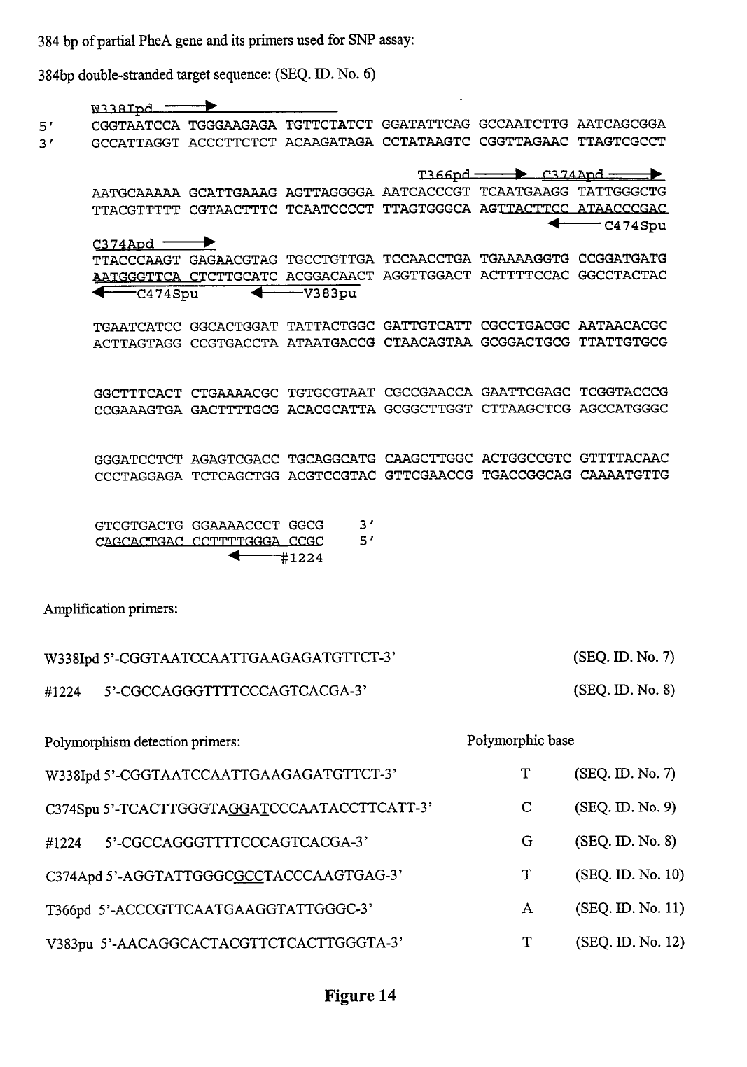

The extension reaction samples contained 0.05 μM of the 384 bp double-stranded DNA, 25 mM ammonium acetate buffer pH 9.3, 1 μM ddNTPs, 2 mM magnesium acetate, and 1 unit of Thermosequenase. Four SNP primers, W338Ipd, C374Spu, #1224, and C374Apd (SEQ. ID. Nos. 7-10), that are capable of annealing to the 384 bp target sequence (Pohnert et al., Biochemistry 38: 12212-17 (1999), which is hereby incorporated by reference, as shown in FIG. 14, were used in individual reactions at 4 μM concentration.

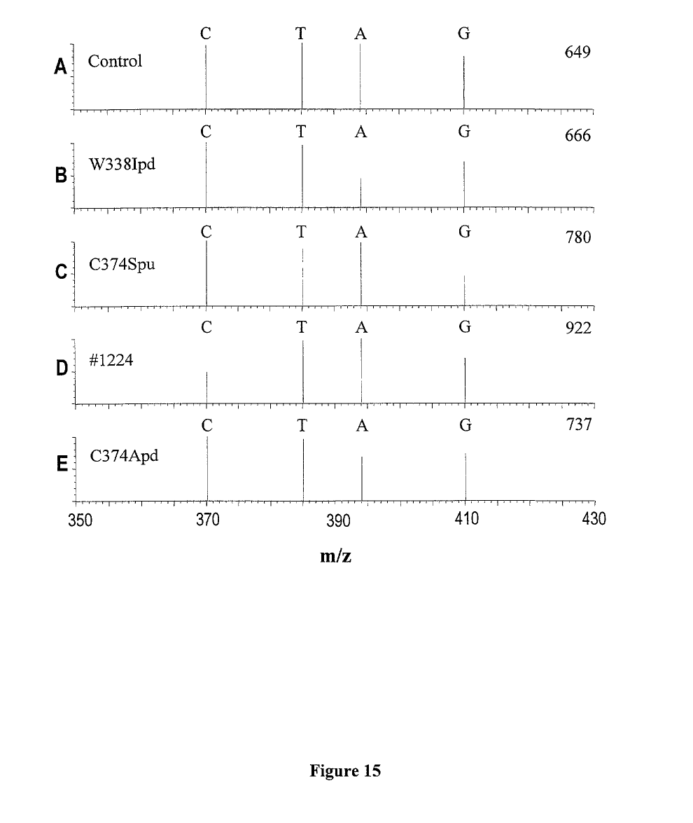

Four different primers were used in individual reactions with the same 384 bp double-stranded DNA template. The extension reactions shown in FIGS. 15B to E each contained 1 μM ddNTPs, 1.25 units of Thermosequenase, 2 mM magnesium acetate, 25 mM ammonium acetate pH 9.3, 4 μM primer, and 0.1 μM 384 bp template. The control reaction was identical to the others except that it did not contain any Thermosequenase. The extension reaction was run for 35 cycles with each cycle composed of a 40 sec denaturing step at 95° C., a 60 sec annealing step at 63° C., and a 60 sec extension step at 72 ° C. The extension reaction samples were prepared for mass spectral analysis by filtering with an Ultrafree-0.5 micron filter unit followed by solid phase extraction using an immobilized iminodiacetic acid gel column. In the reaction shown in FIG. 15B, the primer W338Ipd, having the polymorphic base T, was used. It was observed from the MS/MS spectrum in FIG. 15B that ddATP, m/z 394.1→m/z 79.0, decreased in ion intensity which was expected. The primer C374Spu, was used in the reaction shown in FIG. 15C. This primer has C as its SNP base, so that ddGTP, m/z 410.1→m/z 79.0, was expected to decrease in intensity. In the reaction shown in FIG. 15C, ddGTP was in fact observed to decrease in intensity. In the reaction shown in FIG. 15D, primer #1224 with the polymorphic base G was used. The expected decrease in ddCTP, m/z 370.1→m/z 79.0, was observed. The primer C374Apd was used in the reaction shown in FIG. 15E. This primer has the polymorphic base T, and, therefore, it was expected that ddATP, m/z 394.1→m/z 79.0, would decrease in intensity. This was exactly what was observed in the reaction shown in FIG. 15E. Consequently, this analysis technique works equally well with single and double-stranded DNA. Table 4 shows the peak area ratios of the bases for the control sample compared to the four different SNP primer reactions.

TABLE 4
Summary of the Peak Area Ratios of PCR Extension Reaction Samples
Containing Homogeneous Double-Stranded DNA Template.
Samples were prepared in duplicate.

| | Peak Area Ratios | | | | | |
|---|---|---|---|---|---|---|
| | | | | 385/ | 385/ | 394/ |
| Sample | 370/385 | 370/394 | 370/410 | 394 | 410 | 410 |
| Control - No Enzyme | 0.93 | 0.92 | 1.50 | 0.99 | 1.63 | 1.65 |
| Control - No Enzyme | 0.91 | 0.95 | 1.40 | 1.04 | 1.54 | 1.48 |
| Primer = W338Ipd | 1.28 | 4.58 | 1.99 | 3.53 | 1.56 | 0.44 |
| Primer = W338Ipd | 1.11 | 3.18 | 1.81 | 2.86 | 1.63 | 0.57 |
| Primer = C374Spu | 1.08 | 1.01 | 3.10 | 0.93 | 2.87 | 3.09 |
| Primer = C374Spu | 0.99 | 1.06 | 3.77 | 1.07 | 3.80 | 3.56 |
| Primer = #1224 | 0.24 | 0.22 | 0.39 | 0.91 | 1.61 | 1.78 |
| Primer = #1224 | 0.20 | 0.20 | 0.34 | 1.04 | 1.73 | 1.66 |
| Primer = C374Apd | 1.18 | 2.27 | 2.06 | 1.91 | 1.74 | 0.91 |
| Primer = C374Apd | 0.99 | 1.96 | 1.63 | 1.99 | 1.65 | 0.83 |

Note:
370 denotes the transition m/z 370.1 → m/z 79.0
385 denotes the transition m/z 385.1 → m/z 79.0
394 denotes the transition m/z 394.1 → m/z 79.0
410 denotes the transition m/z 410.1 → m/z 79.0

By comparing the peak area ratios of the control samples to those samples containing enzyme, the SNP bases can be unambiguously identified using double-stranded DNA as a template. All expected results, predicted in FIG. 15, were observed with each base consumed by more than 60%. For example, the primer W338Ipd has the SNP base T, and the concentration of only ddATP was found dramatically reduced, as shown in FIG. 15B, while the other ddNTP bases remained unchanged. Therefore, earlier concerns of reannealing of the two complementary DNA strands competing with the annealing of the primer are unsubstantiated. Once again, the relative standard deviation of each sample and its duplicate was typically less than 15%.

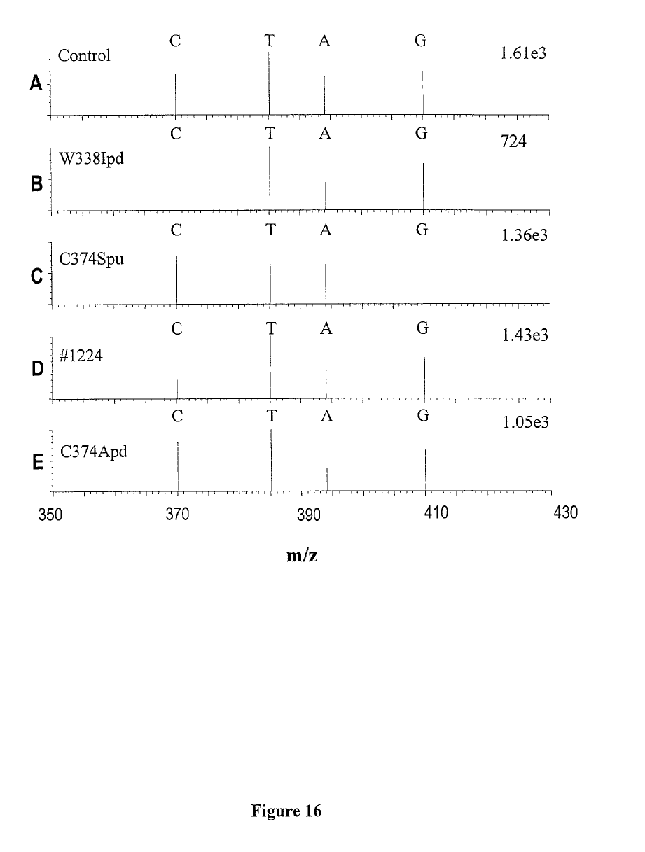

The above set of reactions was repeated, with the exception that the extension reaction samples were not passed through an Ultrafree-0.5 micron filter unit prior to treatment with the iminodiacetic acid gel column. This omission in the sample preparation process lead to an overall increase in sensitivity. In this set of reactions, double-stranded DNA was used as a template. Four different primers were used in individual reactions with the same double-stranded DNA 384 bp template. The extension reactions shown in FIGS. 16B-E each contained 1 μM ddNTPs, 1.25 units of Thermosequenase, 2 mM magnesium acetate, 25 mM ammonium acetate pH 9.3, 4 μM primer, and 0.1 μM 384 bp template. The control reaction was identical to the others except that it did not contain any Thermosequenase. The extension reaction was run for 35 cycles with each cycle composed of a 40 sec denaturing step at 95° C., a 60 sec annealing step at 63° C., and a 60 sec extension step at 72° C. The extension reaction samples were prepared for mass spectral analysis simply by solid phase extraction (SPE) using an immobilized iminodiacetic acid gel column. In the reaction shown in FIG. 16B, the primer W338Ipd, having the polymorphic base T, was used. It was observed from the MS/MS spectrum in FIG. 16B that ddATP, m/z 394.1→m/z 79.0, decreased in ion intensity which was expected. The primer C374Spu, was used in the reaction of FIG. 16C. This primer has C as its SNP base, so that ddGTP, m/z 410.1→m/z 79.0, was expected to decrease in intensity. In the reaction shown in FIG. 16C, ddGTP was in fact observed to decrease in intensity. In the reaction shown in FIG. 16D, primer #1224 with the polymorphic base G was used. The expected decrease in ddCTP, m/z 370.1→m/z 79.0, was observed. The primer C374Apd was used in reaction shown in FIG. 16E. This primer has the polymorphic base T, and, therefore, it was expected that ddATP, m/z 394.1→m/z 79.0, would decrease in intensity. This was exactly what was observed in the reaction shown in FIG. 16E. In this set of reactions, it was determined that filtering prior to the SPE treatment was not necessary and that higher sensitivity was obtained for extension reaction samples that are not filtered. The peak area ratio results of the data shown in FIG. 16 is summarized in Table 5.

TABLE 5
Summary of the Peak Area Ratios of PCR Extension Reaction Samples
Containing Homogeneous Double-Stranded DNA Template.
These samples were not filtered before treatment with IDA columns.

| | Peak Area Ratios | | | | | |
|---|---|---|---|---|---|---|
| | | | | | 385/ | 394/ |
| Sample | 370/385 | 370/394 | 370/410 | 385/394 | 410 | 410 |
| Control | 0.59 | 1.11 | 0.93 | 1.88 | 1.58 | 0.84 |
| Primer = W338Ipd | 0.79 | 51.13 | 1.42 | 64.87 | 1.79 | 0.03 |
| Primer = C374Spu | 0.69 | 1.35 | 3.33 | 1.94 | 4.79 | 2.47 |
| Primer = #1224 | 0.10 | 0.20 | 0.18 | 1.98 | 1.79 | 0.90 |
| Primer = C374Apd | 0.71 | 6.82 | 1.31 | 9.55 | 1.84 | 0.20 |

Note:
370 denotes the transition m/z 370.1 → m/z 79.0
385 denotes the transition m/z 385.1 → m/z 79.0
394 denotes the transition m/z 394.1 → m/z 79.0
410 denotes the transition m/z 410.1 → m/z 79.0

### Example 8—Detection of pheA Gene Mutations

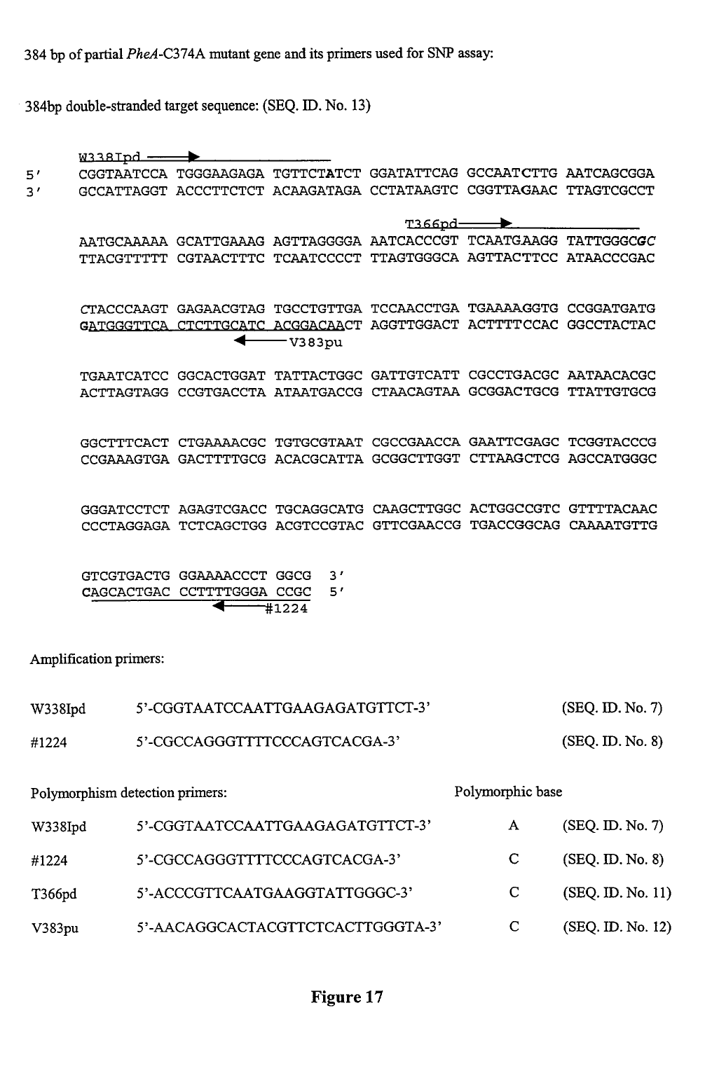

A 384 bp PCR product of partial pheA gene with a C374A mutation (SEQ. ID. No. 13) was constructed by site-directed mutagenesis and amplified by PCR amplification with a mutagenic primer, W338Ipd primer (SEQ. ID. No. 7), as forward primer, #1224 primer (SEQ. ID. No. 8) as reverse primer, and pSZ87 plasmid as a template. The pSZ87 plasmid containing the C374A mutation in the parent vector pJS1 was constructed as described (Pohnert et al., Biochemistry 38: 12212-17 (1999), which is hereby incorporated by reference). The sequence of the double-stranded 384 bp-C374A mutant PCR product is shown in FIG. 17, in which three site-directed mutated bases are shown in italics. The sequence of two amplification primers and two polymorphic detection primers are included. For each primer, the primer binding site to one or the other strand of the target DNA sequence is indicated by a line, and the direction of DNA synthesis is indicated by an arrow. The polymorphic bases for each detection primer are listed and the complementary bases in the target sequence for each detection primer is shown in bold. An equal molar mixture of 384 bp wild type (SEQ. ID. No. 6) and C374A mutant DNA (SEQ. ID. No. 13) is used as a template to further demonstrate this method for detection of heterogeneous polymorphic bases. To identify two SNP bases in the heterogeneous reactions, two additional SNP primers, T366pd (SEQ. ID. No. 11) and V383pu (SEQ. ID. No. 12) were synthesized and used for the heterogeneous assay as shown in FIG. 17.

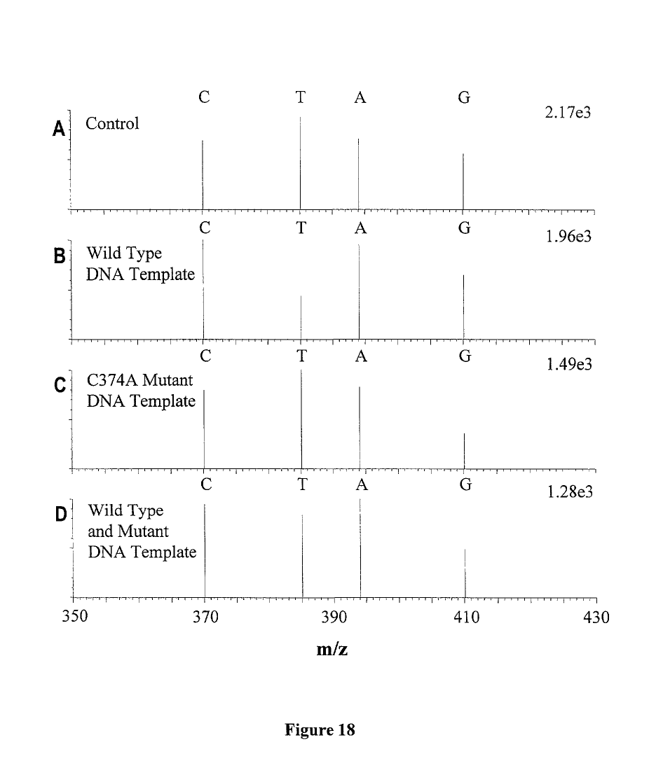

In this set of reactions, T366pd was used as the primer. Two different 384 bp DNA templates were used. The extension reactions each contained 1 μM ddNTPs, 1.25 units of Thermosequenase, 2 mM magnesium acetate, 25 mM ammonium acetate pH 9.3, 4 μM T366pd primer, and 0.12 μM 384 bp template. The control reaction was identical to the others except that it did not contain any Thermosequenase. The results for this reaction are shown in FIG. 18A. The extension reaction was run for 35 cycles with each cycle composed of a 40 sec denaturing step at 95° C., a 60 sec annealing step at 63° C., and a 60 sec extension step at 72° C. The extension reaction samples were prepared for mass spectral analysis simply by solid phase extraction using an immobilized iminodiacetic acid gel column. Filtering prior to the SPE treatment was not performed. In FIG. 18B, wild type 384 bp DNA was used as the template, and, consequently, the polymorphic base was A. The results in FIG. 18B indicate that the expected consumption of free ddTTP occurred. FIG. 18C shows the resulting mass spectrum from a reaction with C374A mutant DNA template. In this example, C becomes the SNP base, and the expected decrease in intensity of ddGTP was observed. FIG. 18D shows the resulting mass spectrum when both templates were added in an equal molar ratio such that the combined concentration of DNA template remained 0.12 μM. This situation closely resembled any heterozygous case that could be encountered. Both polymorphic bases A and C were present in this mixture. The SRM MS/MS mass spectrum of the remaining free ddNTPs after the PCR extension reaction showed that the ion current for both ddTTP and ddGTP decreased in intensity, as predicted. It was calculated that ddTTP and ddGTP were consumed approximately 48% and 38%, respectively. Consequently, this analysis technique can unambiguously identify the polymorphic bases in double-stranded DNA for both homozygous and heterozygous cases.

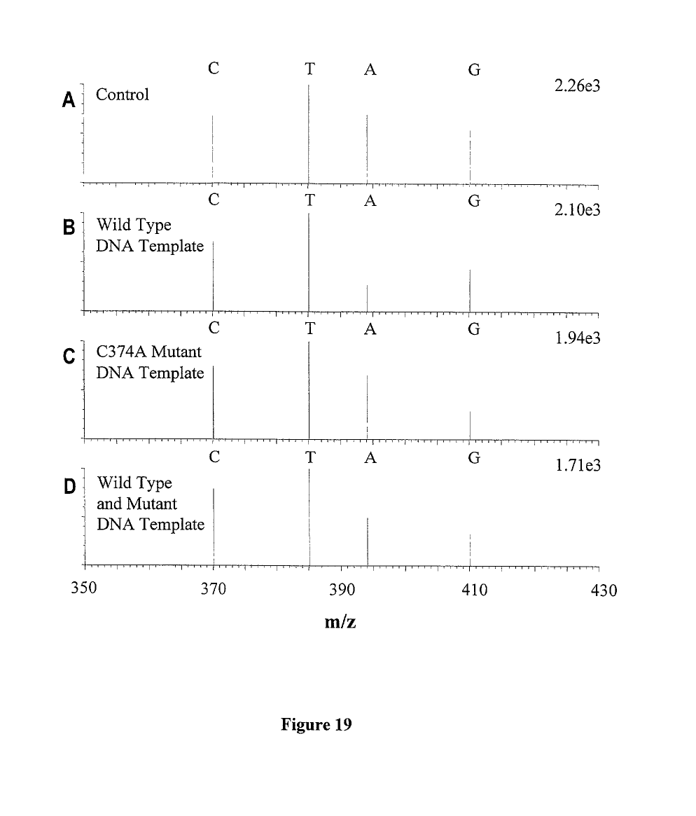

The above reaction steps were repeated with V383pu being used as the primer. Two different 384 bp DNA templates were used. In FIG. 19B, wild type 384 bp DNA was used as the template, and, consequently, the polymorphic base was T and the expected consumption of free ddATP occurred. FIG. 19C shows the resulting mass spectrum from a reaction with C374A mutant DNA template. Here, C became the SNP base, and the expected decrease in intensity of ddGTP was observed. FIG. 19D shows the resulting mass spectrum when both templates were added in an equal molar ratio such that the combined concentration of DNA template remained 0.12 μM. This situation closely resembled any heterozygous case that could be encountered. Both polymorphic bases T and C were present in this mixture. The SRM MS/MS mass spectrum of the remaining free ddNTPs after the PCR extension reaction showed that the ion current for both ddATP and ddGTP decreased in intensity, as predicted. It was calculated that ddATP and ddGTP were consumed approximately 42% and 32%, respectively, in this reaction. Consequently, this analysis technique can unambiguously identify the polymorphic bases in double-stranded DNA for both homozygous and heterozygous cases.

A summary of the mean peak area ratios and standard deviations for the results shown in FIG. 18 and FIG. 19 are listed in Table 6.

TABLE 6
Summary of the Mean Peak Area Ratios ± Standard Deviation of Several
Homogenous and Heterogeneous Double-Stranded DNA Samples.
The statistics are derived from three injections of each of three
replicates of each sample, so that n = 9. The
corresponding relative
standard deviations were less than 15.8%.

| | Mean Peak Area Ratios ± Standard Deviations | | | | | |
|---|---|---|---|---|---|---|
| Sample | 370/385 | 370/394 | 370/410 | 385/394 | 385/410 | 395/410 |
| Control, no enzyme | 0.569 ± 0.029 | 0.871 ± 0.069 | 1.33 ± 0.06 | 1.53 ± 0.07 | 2.33 ± 0.08 | 1.53 ± 0.07 |
| W883Ipd and wild type | 0.499 ± 0.015 | 2.14 ± 0.07 | 1.13 ± 0.07 | 4.29 ± 0.14 | 2.26 ± 0.10 | 0.529 ± 0.029 |
| C3745pu and wild type | 0.663 ± 0.019 | 0.957 ± 0.024 | 4.75 ± 0.20 | 1.44 ± 0.02 | 7.17 ± 0.44 | 4.97 ± 0.28 |
| H1224 and mutant | 0.24 ± 0.030 | 0.307 ± 0.043 | 0.544 ± 0.070 | 1.43 ± 0.06 | 2.55 ± 0.09 | 1.78 ± 0.06 |
| C374Apd and wild type | 0.674 ± 0.020 | 3.29 ± 0.20 | 1.67 ± 0.13 | 4.89 ± 0.43 | 2.49 ± 0.24 | 0.510 ± 0.033 |
| T366pd and wild type | 2.82 ± 0.12 | 1.03 ± 0.04 | 1.65 ± 0.08 | 0.365 ± 0.021 | 0.586 ± 0.033 | 1.61 ± 0.04 |
| V383pu and wild type | 0.672 ± 0.17 | 5.06 ± 0.76 | 2.00 ± 0.21 | 7.52 ± 1.11 | 2.97 ± 0.29 | 0.398 ± 0.038 |
| T366pd and mutant | 0.748 ± 0.049 | 1.01 ± 0.07 | 4.84 ± 0.63 | 1.35 ± 0.07 | 6.49 ± 0.94 | 4.81 ± 0.72 |
| V383pu and mutant | 0.697 ± 0.047 | 1.25 ± 0.10 | 5.45 ± 0.73 | 1.80 ± 0.08 | 7.80 ± 0.68 | 4.34 ± 0.28 |
| T366pd and | 1.25 ± 0.09 | 0.970 ± 0.033 | 3.76 ± 0.29 | 0.778 ± 0.061 | 3.00 ± 0.10 | 3.88 ± 0.30 |
| wild type and mutant | | | | | | |
| V383pu and | 0.711 ± 0.034 | 2.36 ± 0.33 | 4.77 ± 0.45 | 3.33 ± 052 | 6.72 ± 0.79 | 2.04 ± 0.27 |
| wild type and mutant | | | | | | |

Note:
370 denotes the transition m/z 370.1 → 79.0
385 denotes the transition m/z 385.1 → 79.0
394 denotes the transition m/z 394.1 → 79.0
410 denotes the transition m/z 410.1 → 79.0
The data for primer #1224 was acquired on a different day than all the other data.

In addition, the mean +standard deviation is provided for several other samples studied. Table 7 lists the mathematically normalized percent of free dideoxynucleotide bases remaining in solution following primer extension reactions for the results shown in FIG. 18 and FIG. 19.

TABLE 7
Mathematically normalized percent of free dideoxynucleotide bases
remaining following primer extension reactions shown in FIGS. 16 and 17.

| | | | Mean ± |
|---|---|---|---|
| Sample (384 bp template-primer) | Consumed bases | n | SD |
| Homogeneous template | | | |
| Wild type-T366pd | ddTTP | 9 | 23.0 ± 2.5 |
| Wild type-V383pu | ddATP | 9 | 21.4 ± 4.5 |
| C374A mutant-T366pd | ddGTP | 9 | 32.4 ± 4.2 |
| C374A mutant V383pu | ddGTP | 9 | 30.1 ± 5.1 |
| Heterogeneous template | | | |
| Wild type + C374A mutant-T366pd | ddTTP | 6 | 48.1 ± 4.6 |
| | ddGTP | 6 | 37.7 ± 3.3 |
| Wild type + C374A mutant-V383pu | ddATP | 6 | 42.2 ± 7.9 |
| | ddGTP | 6 | 31.8 ± 4.5 |

These results clearly demonstrate the feasibility of using ESI-MS/MS for SNP genotyping by monitoring unreacted dideoxynucleotides remaining in the solution and provide evidence that any known SNP can be analyzed by this technique.

### Example 9—Dependence of ddNTP Consumption on Template Concentration and Number of Thermal Cycles

To optimize the primer extension conditions for the most efficient incorporation of ddNTPs into the SNP primer, a series of primer extension reactions were performed varying both the single-stranded template A (SEQ. ID. No. 1) concentration from 5 to 100 nM and the 384 bp double-stranded DNA (SEQ. ID. No. 6) concentration from 5 to 150 nM. In addition to varying the template concentration, the number of thermal cycles was varied between 10 and 60 cycles for every concentration of template.

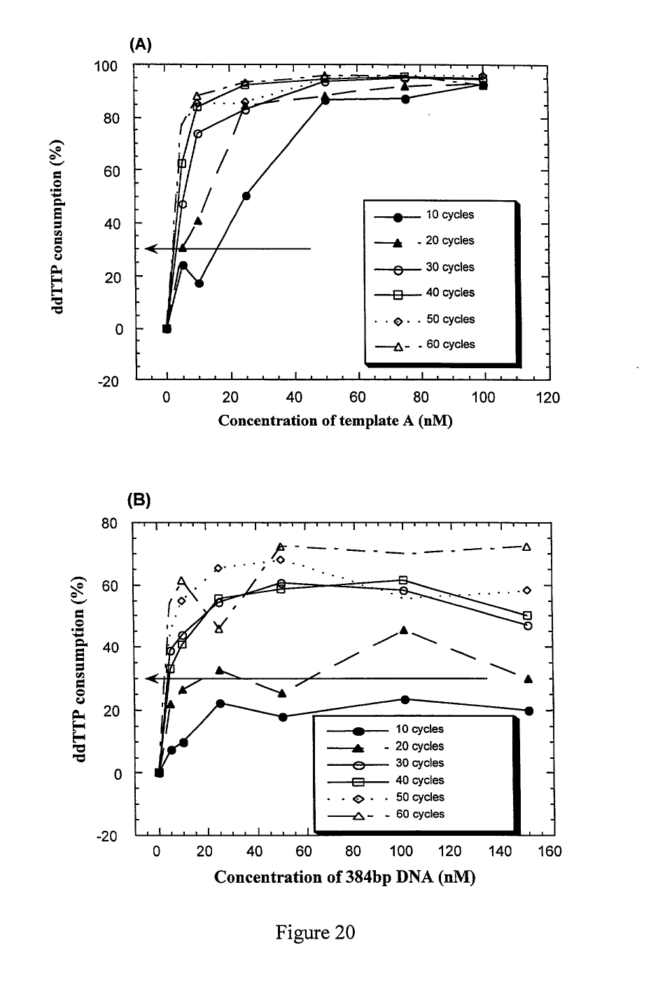

For the single-stranded DNA experiment, template A (5′ CCCCTGTATCCTGTGTGAAATTGTTATCCGCTC 3′, SEQ. ID. No. 1), corresponding to the flanking region of the poly-restriction sites of pUC18/19 plasmid, was used as a target template. The concentration of template was varied at 0 nM, 5 nM, 10 nM, 25 nM, 50 nM, 75 nM, and 100 nM. The universal primer #1233 (SEQ. ID. No. 5) which is a complement to the above synthetic template, was used as the SNP primer at a concentration of 4 μM. The reaction was set up in a total volume of 50 μL, which in addition to the template and primer, was composed of 25 mM ammonium acetate pH 9.3, 1 μM of each ddNTP, 2 mM magnesium acetate, and 1 unit of Thermosequenase. The reaction mixture was subjected to 10, 20, 30, 40, 50, or 60 thermal cycles with each cycle consisting of 95° C. for 30 sec, 60° C. for 60 sec, and 72° C. for 60 sec. The extension reaction samples were prepared for mass spectral analysis by solid phase extraction using an immobilized iminodiacetic acid gel column. The results are displayed in FIG. 20A.

For the double-stranded DNA experiment, a 384 bp PCR product of pheA partial gene (SEQ. ID. No. 6) was used as the template at concentrations of 0 nM, 5 nM, 10 nM, 25 nM, 50 nM, 100 nM, or 150 nM. In addition to the template, the reaction mixture also contained 4 μM of T366pd SNP primer (SEQ. ID. No. 11), 1 μM of each ddNTP, 25 mM ammonium acetate pH 9.3, 2 mM magnesium acetate, and 1-2 units of Thermosequenase. The 50 μL reaction mixture was thermally cycled 10, 20, 30, 40, 50, or 60 times at 95° C. for 30 sec, 63° C. for 60 sec, and 72° C. for 30 sec. The extension reaction samples were prepared for mass spectral analysis by solid phase extraction using an immobilized iminodiacetic acid gel column. The results are displayed in FIG. 20B.

In the primer extension reaction, both the template concentration and the number of thermal cycles are important for adequate incorporation of free ddNTPs into unextended primers. It was determined through these optimization studies that there is a large difference in the ddNTP incorporation rate between extension reactions containing single-stranded DNA template and those containing double-stranded PCR product as template. When single-stranded DNA was used as a template, the following cases permitted the ddNTP to be consumed by at least 30% in the primer extension reaction, thereby allowing the genotype to be scored accurately by ESI/MS: 10 nM template for 20 cycles, 20 nM template for 10 cycles, or 5 nM for 30 cycles. These results are shown in FIG. 20A. FIG. 20B shows that when double-stranded DNA was used as template, 5 nM template for 30 cycles permits accurate scoring.

The primer extension efficiency is lower when double-stranded DNA template is present in the primer extension reaction than when single-stranded DNA template is present, as displayed in FIG. 20. This can be explained by considering the competition that takes place in a primer extension reaction containing double-stranded DNA template arriving between the SNP primer and the complementary strand to hybridize to the template strand. When only single-stranded template is present, the competition is non-existent and, consequently, the primer extension efficiency is higher. This competition is the reason for which the maximum incorporation efficiency is obtained at 50 nM of double-stranded DNA template, using the extension conditions provided. At higher concentrations of double-stranded DNA, the excess template results in self-annealing of the template being more probable than the hybridization of the SNP primer to one stranded of template. As one would expect, increasing the SNP primer concentration from 4 μM to 6 μM increases the incorporation efficiency of reactions containing a high concentration of double-stranded DNA template. Although the incorporation efficiency for double-stranded DNA template is lower than for single-stranded PCR product, the primer extension efficiency was sufficient for a SNP base to be accurately assigned using only 5 nM of double-stranded template. Since typical PCR amplifications produce from 10⁻⁸ M to 10⁻⁷ M of PCR product (Mathieu-Daude et al., Nucleic Acids Res 24: 2080-6 (1996), which is hereby incorporated by reference), the ESI/MS-based SNuPE assay can confidently and unambiguously assign a SNP base from double-stranded DNA template using 20 to 30 primer extension thermal cycles.

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. A method of detecting single nucleotide polymorphisms comprising:
providing a target nucleic acid molecule;
providing an oligonucleotide primer complementary to a portion of the target nucleic acid molecule;
providing a nucleic acid polymerizing enzyme;
providing a plurality of types of nucleotide analogs;
blending the target nucleic acid molecule, the oligonucleotide primer, the nucleic acid polymerizing enzyme, and the nucleotide analogs, each type being present in a first amount, to form an extension solution where the oligonucleotide primer is hybridized to the target nucleic acid molecule to form a primed target nucleic acid molecule and the nucleic acid polymerizing enzyme is positioned to add nucleotide analogs to the primed target nucleic acid molecule at an active site;
extending the oligonucleotide primer in the extension solution by using the nucleic acid polymerizing enzyme to add a nucleotide analog to the oligonucleotide primer at the active site to form an extended oligonucleotide primer, wherein the nucleotide analog being added is complementary to the nucleotide of the target nucleic acid molecule at the active site;
determining the amounts of each type of the nucleotide analogs in the extension solution after said extending, each type being a second amount;
comparing the first and second amounts of each type of the nucleotide analog; and
identifying the type of nucleotide analog where the first and second amounts differ as the nucleotide added to the oligonucleotide primer at the active site so that the nucleotide at the active site of the target nucleic acid molecule is determined.

2. A method according to claim 1, wherein each type of nucleotide analog is a dideoxynucleotide analog.

3. A method according to claim 1, wherein said determining is carried out by electrospraying the extension solution.

4. A method according to claim 3, wherein said electrospraying is carried out with an electrospray device comprising:
a substrate having an injection surface and an ejection surface opposing the injection surface, wherein the substrate is an integral monolith comprising:
an entrance orifice on the injection surface;
an exit orifice on the ejection surface;
a channel extending between the entrance orifice and the exit orifice; and
a recess extending into the ejection surface and surrounding the exit orifice, thereby defining a nozzle on the ejection surface.

5. A method according to claim 4, wherein the electrospray device further comprises:
a voltage application system comprising:
a first electrode attached to said substrate to impart a first potential to said substrate and
a second electrode to impart a second potential, wherein the first and the second electrodes are positioned to define an electric field surrounding the exit orifice.

6. A method according to claim 5, wherein the first electrode is electrically insulated from fluid passing through said electrospray device and the second potential is applied to the fluid.

7. A method according to claim 5, wherein the first electrode is in electrical contact with fluid passing through said electrospray device and the second electrode is positioned on the ejection surface.

8. A method according to claim 5, wherein application of potentials to said first and second electrodes causes fluid passing through said electrospray device fluid to discharge from the exit orifice in the form of a spray.

9. A method according to claim 5, wherein application of potentials to said first and second electrodes causes fluid passing through said electrospray device fluid to discharge from the exit orifice in the form of droplets.

10. A method according to claim 4, wherein said electrospray device further comprises:
a porous polymeric material associated with said electrospray device at a location suitable to effect liquid chromatographic separation of materials passing through said electrospray device.

11. A method according to claim 4, wherein said substrate has a plurality of entrance orifices on the injection surface, a plurality of exit orifices on the ejection surface with each of the plurality of exit orifices corresponding to a respective one of the plurality entrance orifices, and a plurality of channels extending between one of the plurality of exit orifices and the corresponding one of the plurality of entrance orifices.

12. A method according to claim 3, wherein said determining comprises detecting the amounts of each type of the nucleotide analogs in the electrospray.

13. A method according to claim 12, wherein said detecting is carried out by mass spectrometry, fluorescence, ion conductivity, liquid chromatography, capillary electrophoresis, nuclear magnetic resonance, colorimetric ELISA, immunoradioactivity, radioactivity, or combinations thereof.

14. A method according to claim 3 further comprising:
passing the extension solution through a metal chelating resin prior to said electrospraying.

15. A method according to claim 14, wherein the metal chelating resin is a magnesium chelating resin.

16. A method according to claim 14 further comprising:
passing the extension solution through a molecular weight filter prior to said passing the extension solution through a metal chelating agent and
passing the extension solution through a discharge conduit after said passing the extension solution through a metal chelating agent and before said electrospraying.

17. A method according to claim 16, wherein the molecular weight filter, the metal chelating agent, and the discharge conduit are integral.

18. A method according to claim 3 further comprising:
evaporating water from the extension solution, leaving a residue and sonicating the residue.

19. A method according to claim 1, wherein the target nucleic acid molecule is a double stranded DNA molecule.

20. A method according to claim 1 further comprising:
amplifying the target nucleic acid molecule by polymerase chain reaction prior to said blending.

21. An electrospray system comprising:
an electrospray device comprising:
a substrate having an injection surface and an ejection surface opposing the injection surface, wherein the substrate is an integral monolith comprising:
an entrance orifice on the injection surface;
an exit orifice on the ejection surface;
a channel extending between the entrance orifice and the exit orifice; and
a recess extending into the ejection surface and surrounding the exit orifice, thereby defining a nozzle on the ejection surface and
a sample preparation device positioned to transfer fluids to said electrospray device and comprising:
a liquid passage and
a metal chelating resin positioned to treat fluids passing through the liquid passage.

22. An electrospray system according to claim 21, further comprising:
a voltage application system comprising:
a first electrode attached to said substrate to impart a first potential to the substrate and
a second electrode to impart a second potential, wherein the first and the second electrodes are positioned to define an electric field surrounding the exit orifice.

23. An electrospray system according to claim 22, wherein the first electrode is electrically insulated from fluid passing through said electrospray device and the second potential is applied to the fluid.

24. An electrospray system according to claim 22, wherein the first electrode is in electrical contact with fluid passing through said electrospray device and the second electrode is positioned on the ejection surface.

25. An electrospray system according to claim 22, wherein application of potentials to said first and second electrodes causes fluid passing through said electrospray device to discharge from the exit orifice in the form of a spray.

26. An electrospray system according to claim 22, wherein application of potentials to said first and second electrodes causes fluid passing through said electrospray device to discharge from the exit orifice in the form of droplets.

27. An electrospray system according to claim 21, wherein the electrospray device further comprises:
a porous polymeric material associated with said electrospray device at a location suitable to effect liquid chromatographic separation of materials passing through said electrospray device.

28. An electrospray system according to claim 21, wherein said substrate has a plurality of entrance orifices on the injection surface, a plurality of exit orifices on the ejection surface with each of the plurality of exit orifices corresponding to a respective one of the plurality entrance orifices, and a plurality of channels extending between one of the plurality of exit orifices and the corresponding one of the plurality of entrance orifices.

29. An electrospray system according to claim 21, wherein the electrospray device further comprises:
a well positioned upstream of and in fluid communication with the entrance orifice.

30. An electrospray system according to claim 29, wherein the liquid passage of the sample preparation device comprises a discharge conduit from which fluid in the sample preparation device is discharged into the well.

31. A system according to claim 21 further comprising:
a molecular weight filter integral with the metal chelating resin.

32. A system for processing droplets/sprays of fluid comprising:
an electrospray system according to claim 21 and
a device to receive fluid droplets/sprays of fluid from the exit orifice of said electrospray device.

33. A system according to claim 32, wherein said substrate has a plurality of entrance orifices on the injection surface, a plurality of exit orifices on the ejection surface with each of the plurality of exit orifices corresponding to a respective one of the plurality of entrance orifices, and a plurality of channels extending between one of the plurality of exit orifices and the corresponding one of the plurality of entrance orifices, said device to receive fluid droplets/sprays comprising:
a daughter plate have a plurality of fluid receiving wells each positioned to receive fluid ejected from a respective one of the exit orifices.

34. A system according to claim 32, wherein said device to receive fluid detects fluorescence, ion conductivity, liquid chromatography, capillary electrophoresis, mass spectrometry, nuclear magnetic resonance, calorimetric ELISA, immunoradioactivity, radioactivity, or combinations thereof.

35. An electrospray system comprising:
an electrospray device comprising:
a substrate having an injection surface and an ejection surface opposing the injection surface, wherein the substrate is an integral monolith comprising:
an entrance orifice on the injection surface;
an exit orifice on the ejection surface;
a channel extending between the entrance orifice and the exit orifice; and
a recess extending into the ejection surface and surrounding the exit orifice, thereby defining a nozzle on the ejection surface and
a sample preparation device positioned to transfer fluids to said
electrospray device and comprising:
a liquid passage and
a molecular weight filter positioned to treat fluids passing through the liquid passage.

36. An electrospray system according to claim 35, further comprising:
a voltage application system comprising:
a first electrode attached to said substrate to impart a first potential to the substrate and
a second electrode to impart a second potential, wherein the first and the second electrodes are positioned to define an electric field surrounding the exit orifice.

37. An electrospray system according to claim 36, wherein the first electrode is electrically insulated from fluid passing through said electrospray device and the second potential is applied to the fluid.

38. An electrospray system according to claim 36, wherein the first electrode is in electrical contact with fluid passing through said electrospray device and the second electrode is positioned on the ejection surface.

39. An electrospray system according to claim 36, wherein application of potentials to said first and second electrodes causes fluid passing through said electrospray device to discharge from the exit orifice in the form of a spray.

40. An electrospray system according to claim 36, wherein application of potentials to said first and second electrodes causes fluid passing through said electrospray device to discharge from the exit orifice in the form of droplets.

41. An electrospray system according to claim 35, wherein the electrospray device further comprises:
a porous polymeric material associated with said electrospray device at a location suitable to effect liquid chromatographic separation of materials passing through said electrospray device.

42. An electrospray system according to claim 35, wherein said substrate has a plurality of entrance orifices on the injection surface, a plurality of exit orifices on the ejection surface with each of the plurality of exit orifices corresponding to a respective one of the plurality entrance orifices, and a plurality of channels extending between one of the plurality of exit orifices and the corresponding one of the plurality of entrance orifices.

43. An electrospray system according to claim 35, wherein the electrospray device further comprises:
a well positioned upstream of and in fluid communication with the entrance orifice.

44. An electrospray system according to claim 43, wherein the liquid passage of the sample preparation device comprises a discharge conduit from which fluid in the sample preparation device is discharged into the well.

45. A system for processing droplets/sprays of fluid comprising:
an electrospray system according to claim 35 and
a device to receive fluid droplets/sprays of fluid from the exit orifice of said electrospray device.

46. A system according to claim 45, wherein said substrate has a plurality of entrance orifices on the injection surface, a plurality of exit orifices on the ejection surface with each of the plurality of exit orifices corresponding to a respective one of the plurality of entrance orifices, and a plurality of channels extending between one of the plurality of exit orifices and the corresponding one of the plurality of entrance orifices, said device to receive fluid droplets/sprays comprising:
a daughter plate have a plurality of fluid receiving wells each positioned to receive fluid ejected from a respective one of the exit orifices.

47. A system according to claim 46, wherein said device to receive fluid detects fluorescence, ion conductivity, liquid chromatography, capillary electrophoresis, mass spectrometry, or combinations thereof.

48. A reagent composition comprising:
an aqueous carrier;
an oligonucleotide primer;
a mixture of nucleotide analogs of different types;
magnesium acetate;
a buffer, and
a nucleic acid polymerizing enzyme.

49. A reagent composition according to claim 48, wherein an excess of the oligonucleotide primer to nucleotide analogs is present in said composition.

50. A reagent composition according to claim 48, wherein a limited concentration of nucleotide analogs is present in said composition.

51. A reagent composition according to claim 48, wherein the buffer is selected from the group consisting of an ammonium bicarbonate, ammonium acetate buffer, and mixtures thereof.

52. A reagent composition according to claim 48, wherein the nucleic acid polymerizing enzyme lacks 3′-5′ exo-nuclease activity.

53. A reagent composition according to claim 48, wherein the composition comprises:
1-10 μM of the oligonucleotide primer;
0.1-10 μM of each of the nucleotide analogs of different type;
0.5-4 mM of the magnesium acetate;
10-50 mM of the buffer; and
0.1-5 units of the nucleic acid polymerizing enzyme.
